(19) United States
(12) Patent Application Publication
(10) Pub. No.: US 2003/0092012 A1
(43) Pub. Date: May 15, 2003

(54) Methods for detecting a plurality of analytes by chromatography

(76) Inventors: Ahmed Chenna, Sunnyvale, CA (US); Tracy J. Matray, Campbell, CA (US); Vincent S. Hernandez, Brookdale, CA (US); Herbert Hooper, Wellesley, MA (US); Sharat Singh, San Jose, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 10/010,949

(22) Filed: Nov. 9, 2001

(51) Int. Cl. C12Q 1/68; C12P 19/34

(52) U.S. Cl. 435/6; 435/91.2

(57) ABSTRACT

The invention provides a method for detecting a target nucleic acid sequence. The method involves contacting one or more target nucleic acid sequences with a set of tagged probes under conditions sufficient for hybridization of a target nucleic acid sequence with a tagged probe, the tagged probes comprising a mobility modifier attached to a nucleic acid target binding moiety by a bond that is cleavable by a nuclease, the nucleic acid target binding moiety containing at least one bond resistant to said nuclease; treating the tagged probe hybridized to the target nucleic acid with a nuclease under conditions sufficient for cleavage of the nuclease-cleavable bond to release a tag reporter; separating a tag reporter using a chromatographic method, and detecting a tag reporter corresponding to a known target sequence.

This application is related to U.S. application Ser. No. 09/698,846 filed Oct. 27, 2000, which is a continuation-in-part of Ser. No. 09/602,586 filed Jun. 21, 2000, which, with Ser. No. 09/684,386, filed Oct. 4, 2000 are continuations-in-parts of Ser. No. 09/561,579, filed Apr. 28, 2000, which is a continuation-in-part of Ser. No. 09/303,029, filed Apr. 30, 1999, all of which are incorporated herein by reference in their entirety.

### BACKGROUND OF THE INVENTION

This invention relates generally to the field of genome and proteome analysis and, more specifically to methods for detecting multiple analytes using chromatographic methods.

Molecular assays have been developed that can identify and quantitate a single analyte, such as a nucleic acid or protein, in a biological sample. These assays can be used, for example, to detect a known mutation in a gene, an infectious agent, or a protein associated with a disease such as cancer. The need to identify and quantitate many analytes from the same sample has become increasingly apparent in many branches of medicine. For example, it can be desirable to analyze a single sample for the presence of several infectious agents at once, for several genes that are involved in a particular disease, or for several genes that are involved in different diseases.

The full sequencing of the human genome has facilitated methods for comparing all of the genes between different cells or individuals. Different individuals are known to contain single base pair changes, called single nucleotide polymorphisms (SNPs), throughout their genomes. It is believed that there will be about one polymorphism per 1,000 bases, resulting in a large number of differences between individuals. These single nucleotide differences between individuals can result in a wide variety of physiological consequences. For example, the presence of different SNPs in cytochrome P450 genes can predict the ability or inability to metabolize certain drugs. Screening individuals for the presence of multiple SNPs could be used to predict how an individual will respond to a particular drug or treatment.

DNA microarrays are devices that contain thousands of immobilized DNA sequences on a miniaturized surface. Arrays have made the process of detecting several genes from a single sample more efficient. Unfortunately, despite the miniaturization of microarray formats, this method still requires significant amounts of the biological sample. In addition, in microarray methods there is a trade-off between high dynamic range and high sensitivity so that in order to increase dynamic range to detect genes of various abundance levels, there is a concomitant decrease in sensitivity.

Proteomics is the study of proteins expressed in a cell. Although more complex than genomics, proteomic analysis can give a more accurate picture of the state of a cell than genomic analysis. For example, the level of mRNA transcribed from a gene does not always correlate to the level of expressed protein. Therefore, analysis of gene expression alone does not always give an accurate picture of the amount of protein derived from a gene of interest. In addition, many proteins are post-translationally modified and these modifications are often important for activity. The type and level of modification of a protein can not be accurately predicted using genome analysis. Therefore, it is important to study a cell in terms of the proteins that are present. For example, it can be desirable to identify and quantitate all proteins present in a cell from an individual and compare the profile with other cells from the same or different individuals.

Assays for the detection of single proteins using antibody-based assays are available. However, analysis of several proteins simultaneously in the same sample can be more difficult. Two-dimensional gel electrophoresis has been used to study the protein content of a cell. This technique requires an individual gel for each sample and sophisticated software to compare the pattern of protein spots between gels. In addition, it is difficult to detect low abundance proteins using this method and several proteins, such as membrane proteins or proteins of very low or high molecular weight, are not amenable to the analysis.

Another aspect of proteome analysis is the study of protein-protein interactions within a cell. These protein-protein interactions form the basis of biochemical pathways within the cell. Two-hybrid assays have been used to study individual protein-protein interactions. However, this assay requires the cloning of the gene for a protein of interest into expression vectors, which is a labor-intensive process. In addition, two-hybrid assays often have a high rate of false positives where the protein of interest non-specifically interacts with another protein. Furthermore, two hybrid assays require several days to perform due to the growth cycle of the cells explored, which limits the number of assays can be performed at one time.

Thus, there exists a need for methods to identify and quantitate a plurality of analytes, including nucleic acids and proteins, quickly and with high sensitivity, high accuracy, and a large dynamic range. The present invention satisfies this need and provides related advantages as well.

### SUMMARY OF THE INVENTION

The invention provides a method for detecting a target nucleic acid sequence. The method involves contacting one or more target nucleic acid sequences with a set of tagged probes under conditions sufficient for hybridization of a target nucleic acid sequence with a tagged probe, the tagged probes comprising a mobility modifier attached to a nucleic acid target binding moiety by a bond that is cleavable by a nuclease, the nucleic acid target binding moiety containing at least one bond resistant to said nuclease; treating the tagged probe hybridized to the target nucleic acid with a nuclease under conditions sufficient for cleavage of the nuclease-cleavable bond to release a tag reporter; separating a tag reporter using a chromatographic method, and detecting a tag reporter corresponding to a known target sequence.

The invention also provides a method for detecting a target analyte. The method involves contacting one or more target analytes with a set of tagged probes attached to a cleavage-inducing moiety under conditions sufficient for binding of a target analyte with a tagged probe, the tagged probes comprising a mobility modifier attached to a target binding moiety by a cleavable linkage, the cleavable linkage being susceptible to cleavage when the cleavage-inducing moiety is activated by visible light; separating tagged probes bound to a target binding moiety from unbound tagged probes; activating the cleavage-inducing moiety with visible light to release a tag reporter; separating a tag reporter using a chromatographic method, and detecting a tag reporter corresponding to target analyte.

In another embodiment, the method for detecting a target analyte involves contacting one or more target analytes with a set of first and second binding reagents under conditions sufficient for binding of a target analyte with said first and second binding reagents, each of the first binding reagents comprising a cleavage-inducing moiety and a target binding moiety, each of the second binding reagents comprising a tagged probe having a mobility modifier attached to a target binding moiety by a cleavable linkage, the cleavable linkage being susceptible to cleavage when in proximity to an activated cleavage-inducing moiety; activating the cleavage-inducing moiety to release a tag reporter; separating a tag reporter using a chromatographic method, and detecting a tag reporter corresponding to a known target analyte.

The invention also provides a method for identifying a binding partner of a specific binding pair. The method involves incorporating a cleavage-inducing moiety into a first binding partner of a specific binding pair; contacting the first binding partner having an incorporated cleavage-inducing moiety with a set of second binding partners under conditions sufficient for binding, each of the second binding partners comprising a tagged probe having a mobility modifier attached to a target binding moiety by a cleavable linkage, the cleavable linkage being susceptible to cleavage when in proximity to an activated cleavage-inducing moiety; activating the cleavage-inducing moiety to release a tag reporter; separating the tag reporter using a chromatographic method, and detecting a tag reporter corresponding to a known second binding partner of a specific binding pair.

### BRIEF DESCRIPTION OF THE DRAWINGS

FIG. 1 shows the structures of several benzoic acid derivatives that can serve as mobility modifiers.

FIG. 2 shows several mobility modifiers that can be used for conversion of amino dyes into tagged probe phosphoramidite monomers.

FIG. 3 shows a cartoon that depicts multiplexed qualtitation of cell surface receptors.

FIG. 4 shows a method for conjugating a tag moiety to an antibody to prepare a tagged probe, and the reaction of the resulting probe with singlet oxygen to produce a sulfinic acid moiety as the released tag reporter.

### DETAILED DESCRIPTION OF THE INVENTION

This invention is directed to methods for the detection of a wide variety of different analytes in a sample. The present invention employs tagged probes that are separately detectable based on a unique physical characteristic, such as a unique mass or charge-to-mass ratio. The tagged probes can be bound to different analytes and released upon specific binding events for simultaneous detection of multiple different analytes in a single sample. For example, large sets of tagged probes with different masses can be generated in order to detect several analytes simultaneously in one assay. After binding the analyte, the complexes are treated with reagents which cleave off the releasable portion, called a tag reporter. The presence of the released tag reporter can be detected and is indicative of the presence and amount of the analyte in the sample.

In one embodiment, the invention provides a method of detecting a target analyte by contacting one or more target analytes with a set of first and second binding reagents. Each of the first binding reagents contains a cleavage-inducing moiety and each of the second reagents contains a tagged probe having a mobility modifier attached to a target-binding moiety by a cleavable linkage. The cleavable linkage is susceptible to cleavage when in proximity to an activated cleavage-inducing moiety. A binding event between first and second bound reagents, either directly or indirectly to the same analyte is sufficient to bring the linkage and cleavage-inducing moiety in close proximity to result in cleavage and release of the corresponding tag reporter upon activation. The tag reporter is detected and will uniquely identify the target analyte to which it was bound.

Throughout this disclosure several terms have been used interchangeable to describe the same component. For example, “tag reporter”, “electrophoresis tag reporter”, and “e-tag reporter” all refer to the same component. These synonymous terms are listed in the definitions below and it is understood that any of the synonymous terms can be used to describe the component.

In defining the terms below, it is useful to consider the makeup of the “tagged probe” also called “electrophoretic probe,” or “e-tag probe,” as used in practicing the methods of the invention. A probe has four basic components or moieties: (i) an optional detection group or moiety, D (ii) a mobility or mass modifier, M (iii) a target-binding moiety, T, and (iv) a linking group, L, that links the mobility or mass modifier and detection group, if used, to the target-binding moiety. These terms will first be examined in the context of the functioning of the tagged or electrophoretic probes in the invention, then more fully defined by their structural features.

The function of a tagged or an electrophoretic probe in the invention is first to interact with a target, such as a single-stranded nucleic acid, a ligand-binding agent, such as an antibody or receptor, or an enzyme, e.g., as an enzyme substrate. The portion or region of the probe that binds to the target is the “target-binding moiety,” abbreviated “T.” After the target-binding moiety of a tagged or an e-tag probe binds to a target, the linking group of the tagged or electrophoretic probe can be cleaved to release a “tag reporter” or an “e-tag reporter” that has a unique mass-to-charge or charge-to-mass ratio and thus a unique electrophoretic mobility in a defined electrophoretic system, unique chromatographic property on a chromatographic medium, or unique mass as determined in a mass spectrometry system. A tag reporter is sometimes referred to as having a unique mass-to-charge ratio or sometimes as having a unique charge-to-mass ratio. Since both mass and charge are known in this ratio, and one value is the inverse of the other, these terms can be used interchangeably to describe the mass and charge characteristics of a tag reporter. The tag reporter or e-tag reporter is composed of the detection group, if used, mobility or mass modifier, and any residue of the linking group that remains associated with the released tag reporter or e-tag reporter after cleavage. Therefore, the second function of the tagged probe or electrophoretic probe is to release a tag reporter or an e-tag reporter, which can be identified according to its unique and known electrophoretic mobility, chromatographic property or mass.

Sets of tagged probes or electrophoretic probes can be used in several applications of the methods of the invention. Each member of a set of tagged probes or electrophoretic probes has a unique target-binding moiety and an associated “tag moiety” or “e-tag moiety” that imparts to the associated tag reporter or e-tag reporter a unique mass, electrophoretic mobility or chromatographic property by virtue of a unique charge-to-mass ratio. In general, the unique charge-to-mass ratio of a tag moiety or an e-tag moiety is due to the chemical structure of the mobility or mass modifier, since the detection group, if used, and linking-group residue (if any) will be common to any set of tagged or electrophoretic probes. However, it is recognized that the detection group can make unique charge and/or mass contributions to the tag reporters or e-tag reporters as well. For example, a set of tagged probes or electrophoretic probes may be made up of a first subset having a group of mobility or mass modifiers which impart unique electrophoretic mobilities or masses to the subset in combination with a detection group having one defined charge and/or mass, and a second subset having the same group of mobility or mass modifiers in combination with a second detection group with a different charge and/or mass, thus to impart electrophoretic mobilities or masses which are unique among both subsets.

The different target-binding moieties in a set of tagged probes or electrophoretic probes are typically designated “Tj”, where the set of probes contains n members, and each Tj, where j=1 to n, is different. Therefore, each target binding moiety can bind specifically and/or with unique affinities to different targets. A set of tagged probes or electrophoretic probes of the invention includes at least about 2 members, generally at least about 5 members, and more generally at least about 10-100 or 100 or more members. Therefore, it can range, for example, from at least about 2 or more to greater than 100.

A “detection group,” abbreviated “D,” refers to a chemical group or moiety that is capable of being detected by a suitable detection system, or alternatively a chemical group providing means for generating a detection group. Means for generating a detection group may include either incorporation of a reactive group to form a bond with a detectable moiety, or the detection group may be a catalytic moiety capable of catalyzing synthesis of a detection group in an electrophoretic system. A tagged probe does not require a specialized detection group when the released tag reporter will be detected using chromatography, mass spectrometry or electrophoresis. However, a detection group can be used to add mass to the released tag reporter for mass spectrometry analysis or for ease of detection upon electrophoretic or chromatographic separation. Detection groups include fluorescent and chromogenic moieties that can be readily detected during or after chromatographic or electrophoretic separation of molecules, for example, by illuminating the moieties with a light source. For example, fluorescent moieties can be illuminated at an excitation wavelength and detected fluorescence of the moieties can be detected at an emission wavelength.

Exemplary fluorescent moieties include Alexa Fluor Dyes, BODIPY fluorophores, fluorescein, Oregon Green, eosins and erythrosins, Rhodamine Green, tetramethylrhodamine, Lissamine Rhodamine B and Rhodamine Red-X Dyes, Cascade Blue dye, coumarin derivatives, naphthalenes, including dansyl chloride. In addition to a fluorophore, a detection component of a tagged probe can be, for example, a chromophore or an electrochemical compound capable of a detectable reaction in the presence of a redox agent. As noted above, the detection group is typically common among a set or subset of different tagged probes or e-tag probes, but may also differ among probe subsets. A detection group also can contain a radioactive isotope as ³H, ³²P or ¹²⁵I.

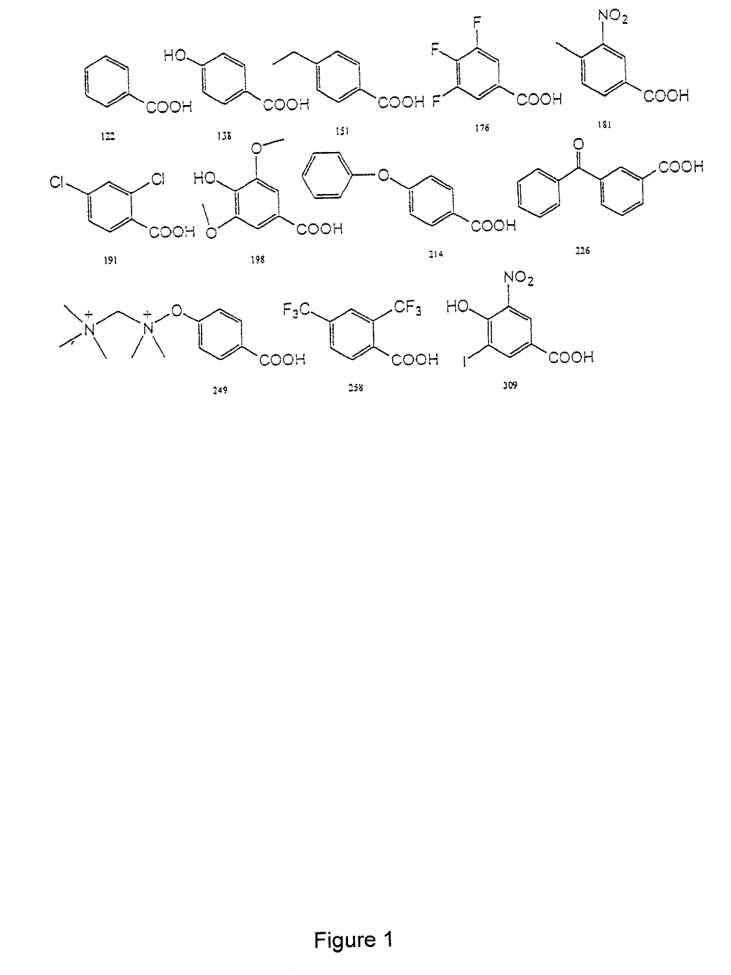

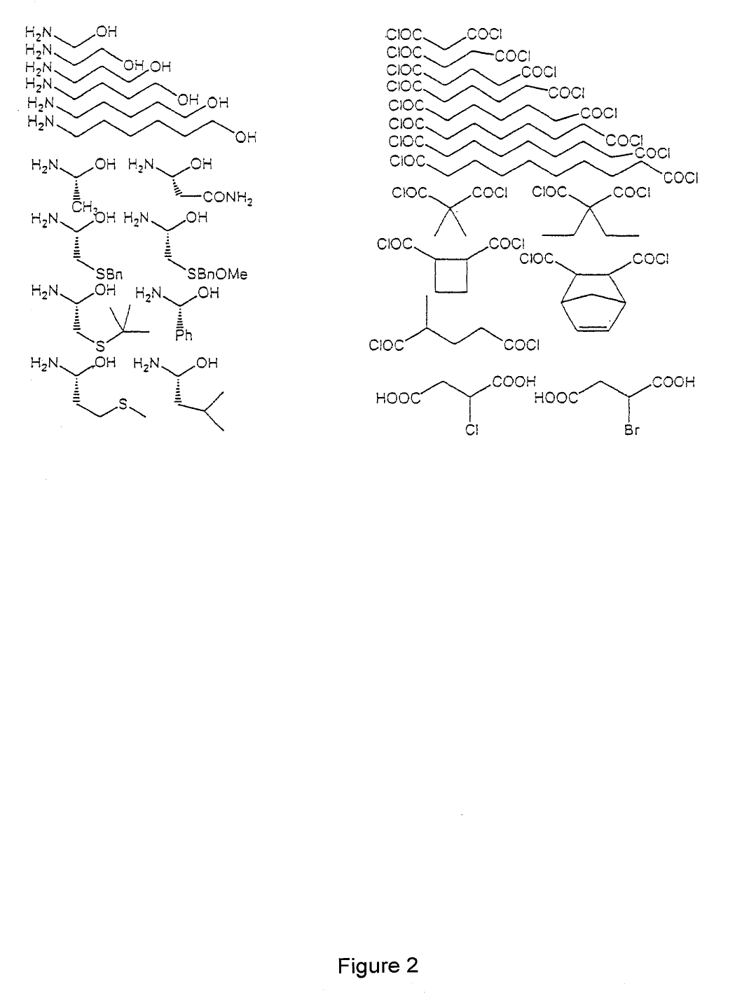

The “mobility modifier,” abbreviated “M,” is a moiety that confers upon the probe or reporter molecule containing it, a “separation characteristic” that allows separation of each probe or reporter molecule from all other probes and reporters of a designated set. The type of separation characteristic used will typically be determined by the separation platform being employed for analysis of an assay. In one preferred embodiment, M is a generally a moiety designed to have a particular charge to mass ratio, and thus a particular electrophoretic mobility in a defined electrophoretic system or a particular chromatographic property in a defined chromatographic system. A mobility modifier can contain a chemical structure that allows a probes or reporters to be distinguished or separated based on a physicochemical property such as molecular size or shape, mass, charge, charge-to-mass ratio, hydrophobicity, and other physicochemical and functional properties, such as affinity for a ligand and behavior on defined chromatographic media. In a set of n tagged probes or electrophoretic probes, each unique mobility modifier is designated Mj, where j=1 to n. FIGS. 1 and 2 depict the structures of several benzoic acid derivative that can serve as mobility modifiers, and several mobility modifiers that can be used for conversion of amino dyes into tagged probe or e-tag phosphoramidite monomers.

The detection group, if used, and the mobility modifier in the tagged probe or electrophoretic probe form a “tag moiety” or an “e-tag moiety” which is linked to the target-binding moiety by a “linking group” which may be only a covalent bond which is cleavable under selected cleaving conditions, or a chemical moiety or chain, such as a nucleotide and associated phosphodiester bond, an oligonucleotide with an internal cleavable bond, an oligopeptide, or an enzyme substrate, that contains a cleavable chemical bond. Cleavage typically occurs as the result of binding of the probe to the target, which is followed by enzyme or catalyzed cleavage of the linking-group bond.

The linking group may or may not contribute a linking-group “residue” to the released tag reporter or e-tag reporter, also dependent on the nature of the linking group and the site of cleavage. For example, where the linking group is a covalent bond, or cleavage of the linking group occurs immediately adjacent the “tag moiety” or “e-tag moiety,” the linking group will leave no residue, i.e., will not contribute additional mass and charge to the released tag reporter or e-tag reporter. Similarly, where the linking group is a chemical group or chain which is cleaved internally or immediately adjacent the target-binding moiety, cleavage of the linking group will leave a residual mass and, possible charge contribution to the released tag reporter or e-tag reporter. In general, this contribution will be relatively small, and will be the same for each different tag reporter or e-tag reporter (assuming a common linking group within the probe set). As such, the residue will not effect the relative electrophoretic mobilities or masses of the released tag reporter or e-tag reporters, nor the ability to resolve the tag reporter or e-tag reporters into mass, electrophoretic, or chromatographic species that can be uniquely identified.

The following definitions are to be understood in the context of the above function of the various components of tagged probes or electrophoretic probes and tag reporters or e-tag reporters. In some case, structure designations based on different lettering schemes are employed, and the equivalency between or among structures with different lettering schemes will be understood by those skilled in the art, in view of the intended function of the structure being referred to.

An electrophoretically tagged probe, or “e-tag probe,” or “tagged probe” refers to one of a set of probes of the type described above having unique target-binding moieties and associated tag moieties or e-tag moieties. The probes are described herein by the following form (D, Mj)-L-Tj, or Mj-L-Tj, wherein according to this terminology, a set of probes will contain n members, where j=1 to n, the detection group is represented by D, Mj is the jth mobility or mass modifier, Tj is the jth target-binding moiety, and the linking group is represented by L. In this structural designation, (D, Mj) intends that either the detection group or the mobility or mass modifier may be the moiety joined to the linking group, i.e., both D-Mj-L-Tj and Mj-D-L-Tj are contemplated.

A “set,” “group” or “library” of tagged probes or electrophoretic probes refers to a plurality of tagged probes or e-tag probes of typically at least five, typically 10-100 or 100 or more probes, each with a unique target-binding moiety and associated tag moiety or e-tag moiety. As used herein, the term “tagged probe set” or “electrophoretic tag probe set” or “e-tag probe set” refers to a set of probes for use in detecting each or any of a plurality of known, selected targets, or for detecting the binding of, or interaction between, each or any of a plurality of ligands and one or more target antiligands.

The term “target-binding moiety” or “T” refers to the component of a tagged probe or an e-tag probe that participates in recognition and specific binding to a designated target. The target-binding moiety may also be defined based on the type of target, e.g., as a SNP detection sequence. In one general embodiment of the target-binding moiety for use in detection of nucleic acid targets, T is an oligonucleotide target-binding moiety. In such cases, T has a sequence of nucleotides U connected by intersubunit linkages:

U1=U2=U3=U4=U5=U6=Ui

where=corresponds to intersubunit linkages Bi, i+1, where i includes all integers from 1 to n, and n is sufficient to allow the oligonucleotide to hybridize specifically with a target nucleotide sequence. Where the target-binding moiety is an oligonucleotide, and enzyme cleavage to release a tag reporter or an e-tag reporter occurs between the first and second 5′ nucleotides (between U1 and U2 above), the linking group and nucleotides forming the target-binding sequence can be expressed by the following representation: U1 is considered the 5′ nucleotide of the target-binding moiety (as in the representation above), and cleavage occurs within this moiety, that is, at a nuclease-susceptible bond between the first and the second nucleotides of the target moiety (between U1 and U2 above). In this representation, the bond between the first and second nucleotides (B1, 2 in the above nomenclature) is the site of cleavage, and all downstream bonds are represented by Bi, i+1, where i is 2 or greater. Typically the penultimate bond will be nuclease-resistant, however the target-binding moiety may include more than one nuclease-resistant linkage adjacent to the nuclease-susceptible linkage, such that cleavage of the probe will yield a single released tag reporter or e-tag reporter species. In this representation, a capture ligand, C, as described further below may be bound to the penultimate nucleotide, U2.

In another exemplary representation, the 5′ nucleotide is designated “N”, and the nuclease-susceptible bond that links it to the 5′ nucleotide (U1) of the target-binding moiety is considered as the linking group. In other words, in this representation, N and all downstream nucleotides are considered as the target-binding region. The same oligonucleotide above would now be expressed as N=U1=U2=U3=U4=U5=U6=Ui, where N is the 5′ nucleotide and participates in target recognition. In this representation, a capture ligand (“C”), can be bound to the ultimate nucleotide (U1).

In another generalized embodiment for use in detection of non-nucleic acid targets, the target-binding moiety, Tj is or includes a ligand capable of binding to or interacting with a target antiligand and L is a linking group connected to Tj by a bond that is cleavable by a selected cleaving agent when the probe is bound to or interacting with the target antiligand. For example, a target-binding moiety can be a polypeptide that binds to another polypeptide or to a nucleic acid. Furthermore, a target-binding moiety can be a polypeptide such as an antibody, or a nucleic acid such as an aptamer.

A “tag reporter” or “electrophoretic tag” or “e-tag reporter” refers to a composition or reagent for unique identification of an entity of interest during separation. A tag reporter or an e-tag reporter has the fundamental structure given as (D, Mj)-L, or Mj-L, where D and Mj are the detection group and the jth mobility or mass modifier, respectively, as defined above, and L is the linking group, and in particular, the bond or residue of the linking group remaining after cleavage. Here, enclosure of D and Mj in parentheses intends that both of the structures D-Mj-L and Mj-D-L are contemplated.

For purposes of clarity, the concept of an electrophoretic tag is consistently referred to herein as “e-tag” or “tag reporter.” As used herein, the term “electrophoretic tag probe” or “e-tag probe” or “tagged probe” refers to a reagent used for target recognition, which comprises an e-tag moiety or tag moiety and a target-binding moiety. Upon interaction with the corresponding target, the e-tag probe or tagged probe undergoes a change resulting in the release of an e-tag reporter or tag reporter. Such an e-tag probe or tagged probe may also be referred to as a binding member.

Tagged probes or e-tag probes of the invention find utility in performing multiplexed assays for detection/analysis of targets including, but not limited to nucleic acid detection, such as sequence recognition, SNP detection, transcription analysis or mRNA determination, allelic determination, mutation determination, HLA typing, MHC determination, and haplotype determination, in addition to detection of other ligands, such as proteins, polysaccharides, etc.

As used herein, the term “tag reporter” or “e-tag reporter” refers to the cleavage product generated as a result of the interaction between a tagged probe or an e-tag probe and its target. In one representation, a tag reporter or an e-tag reporter comprises the tag moiety or e-tag moiety plus a residual portion of the target-binding moiety (Tj) where, as in the nucleotide example, above, one or more nucleotides in the target-binding moiety contain the cleavable linking group. A tag reporter or an e-tag reporter resulting from the interaction of a tagged probe or an e-tag probe and a nucleic acid target typically has the 5′-end terminal nucleotide of a target-binding oligonucleotide.

In another embodiment, the tag reporter or e-tag reporter does not retain any of the target-binding moiety, but may retain a residual portion of the linking group, when the latter is considered separate from the target-binding moiety. Tag reporters or e-tag reporters can be differentiated by electrophoretic mobility or mass and are amenable to electrophoretic separation and detection, although other methods of differentiating the tags such as mass spectrometry may also find use and be preferred in several cases.

A tag reporter or an e-tag reporter resulting from the interaction of a tagged probe or an e-tag probe used to detect the binding of or interaction between a ligand and an antiligand typically has the form (D, Mj)-L′ or Mj-L′. D and Mj are defined above and L′ is the residue of L that remains attached to (D, Mj) after a tag reporter or an e-tag reporter is cleaved from the corresponding tagged probe or e-tag probe.

As used herein, the term “binding event” generally refers to the binding of the target-binding moiety of a tagged probe or an e-tag probe to its target. By way of example, such binding may involve the interaction between complementary nucleotide sequences or the binding between a ligand and target antiligand. In addition, a binding event can refer to the binding of two target analytes such as occurs with a specific binding pair. For example, two polypeptides can specifically bind to each other or a small molecule can bind specifically to a polypeptide.

As used herein, the term “capture ligand”, refers to a group that is typically included within the target-binding moiety or portion of a tagged probe or an e-tag probe, and is capable of binding specifically to a “capture agent” or receptor. The interaction between such a capture ligand and the corresponding capture agent may be used to separate uncleaved tagged probes or e-tag probes from released tag reporters or e-tag reporters. Uncleaved or partially cleaved tagged probes can have one or more chemical groups capable of reacting with or binding to a selected capture agent. The capture ligand can either (i) impart a mass or mobility to probes bound to the capture agent that can be used to distinguish or separate probes within a predetermined range of mass values or electrophoretic mobilities or (ii) immobilize the probes on a solid support. Distinguishing or segregating can include, for example, preventing the bound probes from being separated in a mass spectrometer or migrating during electrophoresis. For example, the probe can contain a capture ligand such as biotin, which is capable of binding specifically to a capture agent such as avidin agarose beads.

As used herein, the terms “analyte,” “target” or “target analyte” are intended to mean any molecule whose presence is to be detected or measured or whose function, interactions or properties are to be studied. Therefore, an analyte includes essentially any molecule for which a detectable probe or assay exists, or can be produced by one skilled in the art. For example, an analyte can be a macromolecule such as a nucleic acid, polypeptide or carbohydrate, or an analyte can be a small organic compound. The presence or absence of an analyte can be measured quantitatively or qualitatively. Analytes can come in a variety of different forms including, for example, simple or complex mixtures, or in substantially purified forms. For example, an analyte can be part of a sample that contains other components or can be the sole or major component of the sample. Therefore, an analyte can be a component of a whole cell or tissue, a cell or tissue extract, a fractionated lysate thereof or a substantially purified molecule. Also an analyte can have either a known or unknown sequence or structure.

Analytes can be monovalent (monoepitopic) or polyvalent (polyepitopic), for example, monovalent analytes include drugs, metabolites, enzyme substrates, enzyme inhibitors, low molecular weight peptides, pesticides, pollutants, and the like. These analytes can generally be from about 100 daltons (D) to about 2,000 D molecular weight, more usually from about 125 D to about 1,000 D molecular weight. However monovalent analytes can also be smaller than 100 D or larger than 1000 D. Polyvalent analytes can include nucleic acids, for example, m-RNA, r-RNA, t-RNA, DNA, DNA-RNA duplexes as well as other forms of nucleic acids well known to those skilled in the art, and poly(amino acids), for example, polypeptides and proteins, peptides, polysaccharides, and combinations thereof. The polyepitopic analytes, to which the subject invention can be applied, can have a large range of molecular weights. For example, in the poly(amino acid) category, the poly(amino acids) of interest will generally be from about 5,000 D to about 5,000,000 D or more molecular weight, and more usually from about 20,000 D to about 1,000,000 D molecular weight. Polyepitopic analytes also can exhibit molecular weights smaller than about 5,000 as well as larger than about 5,000,000 D.

An analyte can be a molecule found directly in a sample such as biological tissue, including body fluids, from a host. Biological tissue includes, for example, excised tissue from an organ or other body part of a host and body fluids, for example, urine, blood, plasma, serum, saliva, semen, stool, sputum, cerebral spinal fluid, tears, mucus, and the like. In addition, a sample can be derived from the environment, for example, air, water, dirt, or from biological materials which are synthetically produced such as libraries of nucleic acids or organic molecules. The sample can be examined directly or can be pretreated to render the analyte more readily detectable. Protein analytes can be released from cells, for example, by lysing the cells and can be isolated using precipitation, extraction, and chromatography. Furthermore, an analyte of interest can be determined by detecting an agent probative of the analyte of interest such as a specific binding pair member complementary to the analyte of interest, whose presence will be detected only when the analyte of interest is present in a sample. Therefore, in such indirect measurements, an agent probative of an analyte becomes the analyte that is detected in an assay.

As used herein, the term “cleavage-inducing moiety” is intended to mean an agent that acts upon a cleavable linkage, or any agent that can produce an agent that acts upon a cleavable linkage, and severs a bond of the cleavage linkage. The cleavage-inducing moiety can be, for example, an enzyme such as a nuclease or protease that can server a phosphodiester or amide bond, respectively. In addition, for example, a cleavage-inducing agent can be an agent that produces singlet oxygen wherein the singlet oxygen is capable of cleaving a susceptible bond within the linkage group. A cleavage-inducing moiety can be added in bulk to a solution that contains a tagged probe with a cleavable linker or a cleavage-inducing moiety be attached or in close proximity to the cleavage-inducing moiety. For example, a cleavage-inducing moiety can be an agent that acts upon a cleavable linkage in a second reagent and thereby potentiates the release of a portion of the second reagent and the released portion is detected.

The nature of the cleavage-inducing moiety that is, or produces, an agent that acts upon a cleavable linkage is dependent on the nature of the cleavable linkage so that they are compatible pairs. For example, a nuclease as a cleavage-inducing moiety and a nuclease-sensitive bond, such as a phosphodiester bond in a nucleic acid sequence, are compatible pairs since the nuclease can cleavage the nuclease-sensitive bond. In addition, a cleavage-inducing moiety can produce an agent and that agent is paired with a bond that is cleavable by the agent. For example, a sensitizer can produce singlet oxygen and then singlet oxygen can cleave a thioether bond.

A cleavage-inducing moiety can be an active species such as, for example, a chemical species that exhibits relatively short-lived activity. Illustrative species include singlet oxygen, hydrogen peroxide, NADH, and hydroxyl radicals, phenoxyradical, superoxide, and the like. Singlet oxygen can be generated from oxygen by dye-sensitized photoexcitation. Singlet oxygen can also be produced by non-photochemical means. One means is by the reaction between hydrogen peroxide and sodium hypochlorite or sodium molybdate. Another means is by reaction between ozone and triphenyl phosphite. A third means is by the reaction between triethylsilane and ozone.

The cleavage-inducing moiety can be a compound that upon activation produces energy as the active agent where energy transfer results in the cleavage of the cleavable linkage. For example, with a Norrish type 2 reaction of o-nitrobenzyl ethers, or anthracene derivatives, upon excitation with light, the energy is dissipated by cleavage of a bond, rather than emission of light or heat.

One particular embodiment of a cleavage-inducing moiety includes a “sensitizer” which is a class of chemical moiety that can produce a short-lived active species such as, for example, singlet oxygen. Therefore, a sensitizer is a molecular class of compounds or reactants that can generate reactive intermediates. Generally, a sensitizer is a photosensitizer. However, other sensitizers can be employed in the present invention including, for example, chemi-activated sensitizer, such as enzymes and metal salts and other substances and compositions that can produce reactive intermediates with or without activation by an external light source. Specific examples of such other substances include, molybdate (MoO4=) salts and chloroperoxidase and myeloperoxidase plus bromide or chloride ion (Kanofsky, J. Biol. Chem. 259:5596 (1983)) which catalyze the conversion of hydrogen peroxide to singlet oxygen and water. For the above examples of sensitizers, hydrogen peroxide can be included as an ancillary reagent, chloroperoxidase can be bound to a surface and molybdate can be incorporated in the aqueous phase of a liposome, respectively. Other sensitizers included within the scope of the invention are compounds that are not true sensitizers but which on excitation by heat, light, ionizing radiation, or chemical activation will release a molecule of singlet oxygen. The members of this class of compounds include the endoperoxides such as 1,4-biscarboxyethyl-1,4-naphthalene endoperoxide, 9,10-diphenylanthracene-9,10-endoperoxide and 5,6,11,12-tetraphenyl naphthalene 5,12-endoperoxide. Heating or direct absorption of light by these compounds releases singlet oxygen.

Photosensitizers are sensitizers for generation of singlet oxygen by excitation with light or other source of irradiation and include, for example, dyes and aromatic compounds. General characteristics of such compounds include, for example, covalently bonded atoms, usually with multiple conjugated double or triple bonds. The compounds generally absorb light in the wavelength range of about 200 to about 1,100 nm, usually, about 300 to about 1,000 nm, preferably, about 450 to about 950 nm, with an extinction coefficient at its absorbance maximum greater than about 500 M⁻¹ cm⁻¹, preferably, about 5,000 M⁻¹ cm⁻¹, more preferably, about 50,000 M⁻¹ cm⁻¹, at the excitation wavelength. The lifetime of an excited state produced following absorption of light in the absence of oxygen will usually be at least about 100 nanoseconds, preferably, at least about 1 millisecond. In general, the lifetime is sufficiently long to permit cleavage of a linkage in a reagent in accordance with the present invention. The photosensitizer excited state usually has a different spin quantum number (S) than its ground state and is usually a triplet (S=1) when the ground state, as is usually the case, is a singlet (S=0). Generally, the photosensitizer has a high intersystem crossing yield. That is, photoexcitation of a photosensitizer usually produces a triplet state with an efficiency of at least about 10%, desirably at least about 40%, generally greater than about 80%.

Photosensitizers chosen are relatively photostable and, generally, do not react efficiently with singlet oxygen. Several structural features are present in most useful photosensitizers. Most photosensitizers have at least one and frequently three or more conjugated double or triple bonds held in a rigid, frequently aromatic structure. They will frequently contain at least one group that accelerates intersystem crossing such as a carbonyl or imine group or a heavy atom selected from rows 3-6 of the periodic table, especially iodine or bromine, or they can have extended aromatic structures.

Photosensitizers can include, for example, benzophenone, 9-thioxanthone, eosin, 9,10-dibromoanthracene, methylene blue, metallo-porphyrins, such as hematoporphyrin, phthalocyanines, chlorophylls, rose bengal, buckminsterfullerene, etc., and derivatives of these compounds having substituents of 1 to 50 atoms for rendering such compounds more lipophilic or more hydrophilic and/or as attaching groups for attachment, for example, to a target-binding moiety. Examples of other photosensitizers that can be utilized in the present invention are those that have the above properties and which can be found enumerated in, for example, N. F. Turro, “Molecular Photochemistry” page 132, W. A. Benjamin Inc., N.Y. 1965. Other sensitizers for generation of singlet oxygen are discussed in, for example, Ullman, et al., Proc. Natl. Acad. Sci. USA 91, 5426-5430 (1994). Examples of combinations that find use in this invention can be found in U.S. Pat. Nos. 5,536,498; 5,536,834; and references cited therein; H. H. Wasserman and R. W. Murray, “Singlet Oxygen,” Academic Press, New York (1979); A. L. Baumstark, “Singlet Oxygen,” Vol. 2, CRC Press Inc., Boca Raton, Fla. 1983.

A sensitizer reagent generally contains a sensitizer and, where the sensitizer is not otherwise able to be associated with the analyte, a binding partner for the analyte, which is usually a member of a specific binding pair, or an analyte analog. The binding partner usually has a high affinity for the analyte. Usually, the binding affinity will be at least about 10⁻⁷M⁻¹, more usually, at least about 10⁻⁸M⁻¹. In one embodiment, the binding partners are receptors, which include antibodies, IgA, IgD, IgG, IgE and IgM and subtypes thereof, enzymes, lectins, nucleic acids, nucleic acid binding proteins, or any other molecule that provides the desired specificity for the analyte in the assay. The antibodies can be polyclonal or monoclonal or mixtures of monoclonal antibodies depending on the nature of the target composition and the targets.

For the cognate cleavable linkage, there are a large number of different functional entities that are stable under the conditions used for binding events with a binding compound that can then be cleaved without adversely affecting the tag reporter. Functional entities can be cleaved by chemical or physical methods, involving oxidation, reduction, solvolysis, for example, hydrolysis, photolysis, thermolysis, electrolysis, and chemical substitution. Specific functional entities include, for example, thioethers that can be cleaved with singlet oxygen, disulfide that can be cleaved with a thiol, diketones that can be cleaved by permanganate or osmium tetroxide, β-sulfones, tetralkylammonium, trialkylsulfonium, tetralkylphosphonium, where the α-carbon is activated with carbonyl or nitro, that can be cleaved with base, quinones where elimination occurs with reduction, substituted benzyl ethers that can be cleaved photolytically, carbonates that can be cleaved thermally, metal chelates, where the ligands can be displaced with a higher affinity ligand, as well as many other functional entities that are known in the literature. Cleavage methods are described, for example, in U.S. Pat. Nos. 5,789,172 and 6,001,579 and references cited therein. Other labile groups can be used as alternatives to moieties cleavable by reaction with singlet oxygen such as those disclosed in, for example, Brown, Contemporary Organic Synthesis 4(3):216-237 (1997), and as will be apparent to one skilled in the art.

Association of a cleavage-inducing moiety with an analyte can be accomplished in a variety of ways, for example, the cleavage-inducing moiety can be associated with the analyte through a target-binding moiety. A cleavage-inducing moiety linked to a target-binding moiety is called a “cleavage-inducing reagent” and is described further below. However, the cleavage-inducing moiety also can be associated with an analyte in the absence of a target-binding moiety in the cleavage-inducing reagent. For example, the cleavage-inducing moiety can be associated directly with the analyte either by attachment, incorporation, absorption, dissolution, surface adsorption, and the like. In one example, a cleavage-inducing moiety can be incorporated into a cell membrane, for example, to study cellular proteins and their interactions or intercalated into a polynucleotide duplex. A cleavage-inducing moiety can be incorporated into a binding partner that binds to a tagged probe.

A “cleavage-inducing reagent” generally consists of two components, a target-binding moiety and a cleavage-inducing moiety. The target-binding moiety for the cleavage-inducing reagent is chosen such that positioning of the cleavage-inducing reagent in close proximity to a tagged probe is dependent on the presence of analyte. The target-binding moiety can be, for example, a binding partner for the analyte that directly binds to the analyte, or alternatively, an analyte analog that binds to a binding partner for the analyte. The nature of the target-binding moiety in the cleavage-inducing reagent depends on the nature of the assay to be conducted, for example, competitive or sandwich, and so forth.

Attachment of a target-binding moiety to the cleavage-inducing moiety can be direct or indirect, covalent or non-covalent and can be accomplished by well-known techniques, commonly available in the literature. See, for example, “Immobilized Enzymes,” Ichiro Chibata, Halsted Press, New York (1978) and Cuatrecasas, J. Biol. Chem., 245:3059 (1970). A wide variety of functional groups are available or can be incorporated. Functional groups include carboxylic acids, aldehydes, amino groups, cyano groups, ethylene groups, hydroxyl groups, mercapto groups, and the like. The manner of linking a wide variety of compounds is well known and is amply illustrated in the literature (see above). The length of a linking group to a target-binding moiety can vary widely, depending upon the nature of the compound being linked, the effect of the distance on the specific binding properties and the like.

The cleavage-inducing reagent can be pre-formed or formed in situ. In the former circumstance the cleavage-inducing reagent has all of its components bound together prior to use in the present methods. In the latter situation at least some of the components of the cleavage-inducing reagent are added separately to a medium in which the present methods are conducted. In one approach the binding partner for the analyte, which is one component of the cleavage-inducing reagent, is added to the medium to bind to analyte if present in the medium. The binding partner comprises a moiety for attachment of the cleavage-inducing moiety of the cleavage-inducing reagent. Usually, this involves a second moiety, which is present on the cleavage-inducing moiety, where the second moiety and the moiety of the binding partner interact providing for attachment of the sensitizer to the binding partner and formation of the cleavage-inducing reagent in situ. Typically, the moieties interact by non-covalent attachment. This situation is exemplified by one of the two moieties comprising a small molecule and the other of the moieties comprising a binding partner for the small molecule. For example, the small molecule can be biotin, digoxin, fluorescein, dinitrophenol, and so forth, and the binding partner for the small molecule is, respectively, avidin, antibody for digoxin, antibody for fluorescein, antibody for dinitrophenol, and so forth.

It can be desirable to have multiple cleavage-inducing moieties attached to a target-binding moiety to increase, for example, the number of active species generated. Where the target-binding moiety has a plurality of sites for attachment such as, for example, a poly(amino acid), such as an antibody, there are a plurality of binding sites on the poly(amino acid) for attachment of cleavage-inducing moieties. To further enhance the number of cleavage-inducing moieties, a hub molecule or nucleus can be employed. The hub nucleus is a polyfunctional material, normally polymeric, having a plurality of functional groups, e.g., hydroxy, amino, mercapto, carboxy, ethylenic, aldehyde, etc., as sites for linking. The functionalities on the hub should be those that are reactive with a functionality on the cleavage-inducing moiety or the target-binding moiety to be attached.

In certain embodiments the cleavage-inducing reagent comprises a support with which one of the components of the cleavage-inducing reagent is associated. The support can be comprised of an organic or inorganic, solid or fluid, water insoluble material, which can be transparent or partially transparent. The support can have any of a number of shapes, such as particle including bead, film, membrane, tube, well, strip, rod, and the like. For supports in which a sensitizer is incorporated, the surface of the support is, preferably, hydrophilic or capable of being rendered hydrophilic and the body of the support is, preferably, hydrophobic. The support can be suspendable in the medium in which it is employed. Examples of suspendable supports, by way of illustration and not limitation, are polymeric materials such as latex, lipid bilayers, oil droplets, cells and hydrogels. Other support compositions include glass, metals, and polymers, either used by themselves or in conjunction with other materials. Binding of target binding moieties to the support can be direct or indirect, covalent or non-covalent and can be accomplished by well-known techniques, commonly available in the literature as discussed above. See, for example, “Immobilized Enzymes,” Ichiro Chibata, supra. The surface of the matrix can be polyfunctional or be capable of being polyfunctionalized or be capable of binding to a target-binding moiety, or the like, through covalent or specific or non-specific non-covalent interactions.

The invention provides methods for identifying a target analyte. The methods of the invention are advantageous for detecting multiple analytes simultaneously in a single sample. Large sets of tagged probes can be generated that allow for the simultaneous detection of multiple analytes. As described further above, a tagged probe generally has a mobility or mass modifying moiety, a target-binding moiety, and a cleavable linking group that links the mobility or mass modifier to the target-binding moiety. After binding to a target analyte, a unique tag reporter is cleaved from the tagged probe and the tag reporter identifies the tagged probe it originates from.

Each released tag reporter has a unique physical characteristic that allows it to be uniquely identified when compared to other tag reporters used in the same assay. The tag reporters can be separated and identified based on this difference. For example, tag reporters can differ from each other based on a unique mass or a unique charge or a unique mass-to-charge ratio. Methods for separating and identifying tag reporters based on these physical differences include, for example, electrophoresis, chromatography, and mass spectrometry.

Electrophoresis is a convenient technique for separating tag reporters. Each tag reporter will have a different mobility through the gel based on its unique mass and charge characteristics. Although not required, a tag reporter detected by electrophoresis can have a detection group or moiety, such as a fluorophore, attached to aid in detection. Fluorescently labeled tag reporters can be separated and identified, for example, using the same gel electrophoresis and detection system used for automated sequencing.

Mass spectrometry can also be used to separate and identify tag reporters. Tag reporters can be ionized in a mass spectrometer and the ions separated in space or time based on their mass-to-charge ratio. The mass spectrometer then calculates a mass associated with each ion. Therefore, when referring to mass spectrometry, the term mass can be used for simplicity to describe a mass-to-charge ratio.

Tag reporters can also be separated using chromatographic methods. Different tag reporters can have different behaviors on chromatographic media based on their unique mass, charge, mass-to-charge ratio, molecular size and shape, hydrophobicity, affinity for a ligand, and other physicochemical and functional properties that influence the interaction of a tag reporter with a particular chromatographic media.

A chromatographic method is used to separate tag reporters based on their chromatographic properties. A chromatographic property can be, for example, a retention time of a tag reporter on a specific chromatographic medium under defined conditions, or a specific condition under which a tag reporter is eluted from a specific chromatographic medium. A chromatographic property of a tag reporter can also be an order of elution, or pattern of elution, of a tag reporter contained in a group or set of tag reporters being chromatographically separated using a specific chromatographic medium under defined conditions. A chromatographic property of a tag reporter is determined by the physical properties of the tag reporter and its interactions with a chromatographic medium and mobile phase. Defined conditions for chromatography include particular mobile phase solutions, column geometry, including column diameter and length, flow rate, pressure and temperature of column operation, and other parameters that can be varied to obtain the desired separation of tag reporters. A tag reporter, or chromatographic property of a tag reporter, can be detected using a variety of chromatography methods.

A chromatographic method useful for separating tag reporters characterized by a variety of different physicochemical properties and structures is liquid chromatography. Methods and reagents useful for performing liquid chromatography separation of a wide range of molecules and molecular complexes are well known to those skilled in the art and are described, for example, in Millner, “High Resolution Chromatography: A Practical Approach”, Oxford University Press, New York (1999), Chi-San Wu, “Column Handbook for Size Exclusion Chromatography”, Academic Press, San Diego (1999), and Oliver, “HPLC of Macromolecules: A Practical Approach, Oxford University Press”, Oxford, England (1989).

Although standard liquid chromatography methods can be used to separate tag reporters, high pressure (or performance) liquid chromatography (HPLC) provides the advantages of high resolution, increased speed of analysis, greater reproducibility, and ease of automation of instrument operation and data analysis. HPLC methods also allow separation of tag reporters based on a variety of physiochemical properties. Tag reporters having similar properties can be used together in the same experiment since HPLC can be used to differentiate between closely related tags. The high degree of resolution achieved using HPLC methods allows the use of large sets of tagged probes because the resulting tag reporters can be distinguished from each other. The ability to detect large sets of tagged probes is an advantage when performing multiplexed detection of target nucleic acids and target analytes.

Sets of tag reporters detected in a single experiment generally are a group of chemically related molecules that differ by mass, charge, mass-charge ratio, detectable tag, such as differing fluorophores or isotopic labels, or other unique characteristic. Therefore, both the chemical nature of the tag reporter and the particular differences among tag reporters in a group of tag reporters can be considered when selecting a suitable chromatographic medium for separating tag reporters in a sample.

Separation of tag reporters by liquid chromatography can be based on physical characteristics of tag reporters such as charge, size and hydrophobicity of tag reporters, or functional characteristics such as the ability of tag reporters to bind to molecules such as dyes, lectins, drugs, peptides and other ligands on an affinity matrix. A wide variety of chromatographic media are suitable for separation of tag reporter based on charge, size, hydrophobicity and other chromatographic properties of tag reporters. Selection of a particular chromatographic medium will depend upon the properties of tag reporters employed.

Separation of tag reporters based on charge can be performed by ion exchange chromatography. Methods for separating peptides, proteins, oligonucleotides, and nucleic acids are well known to those skilled in the art and are described, for example, in Millner, supra (1999). In this technique, separation is based on the exchange of ions (anions or cations) between the mobile phase and ionic sites on the stationary phase. Charged chemical species are covalently bound to the surface of the stationary phase to prepare an ion exchange resin. The mobile phase contains a large number of counterions that are opposite in charge to the resin ionic group to form an ion-pair. A tag reporter having the same ionic charge as the counterion will be in equilibrium with the counterion. The tag reporter ion can exchange with the counter ion to pair with the covalently attached charge on the support. When the tag reporter ion is paired with the charged group on the support, it does not move through the column. Tag reporter ion retention is based on the affinity of different ions on the support and other solution parameters including counterion type, ionic strength and pH.

Ion exchange media fall into two classes that include strong ion exchangers and weak ion exchangers. The charge of weak ion exchangers varies with pH of the mobile phase, while the charge of strong ion exchangers is essentially independent of pH. In most cases, it is advantageous to select a strong exchanger to separate tag reporters, but when tag reporters bind very tightly to strong exchangers, a weak exchanger is advantageous to allow maximum recovery of tag reporters.

Ion exchange media useful for separating tag reporters include both anion or cation exchangers. The choice of whether to use an anion or cation exchanger to separate tag reporters will therefore depend on the charge of the tag reporters at the pH of the chromatographic step. The choice of the pH for the separation can be selected by determining the isolelectric point (pI) of the tag reporter, or the average isoelectric point of a group of tag reporters, and generally using one pH unit above the pI for anion exchange or one pH unit below the pI for cation exchange.

Cation exchange resins have anionic functional groups such as —SO3-, —OPO3- and —COO— and anion exchange matrices usually contain the cationic tertiary and quaternary ammonium groups, with general formulae —NHR2+ and —NR3+. Exemplary ion exchange chromatography media for separating tag reporters that are peptides, polypeptides, nucleic acids and chemical compounds include strong and weak anion and cation exchange resins having functional groups such as sulfonic acid, quaternary amine and tertiary amine, commonly known as S, Q, and DEAE resins, respectively.

Exemplary commercial preparations for separating nucleic acid molecules include Nucleosil SA and Partisil SCX, which are S type resins, Vydac 3040L and Nucleogen 60-7, 500-7, and 4000-7, which are DEAE type resins. Exemplary commercial preparations for separating peptides and polypeptides include Aquapore CX-300 and RP-300.

Separation of tag reporters based on size can be performed by size exclusion chromatography. Separation with this technique is based on the molecular size of the tag reporter in solution. The stationary phase generally is gel with an inert porous surface. Large tag reporters in the solute, which cannot enter the pores and are not retained by the column, elute first. Smaller tag reporters, capable of permeating all of the pores, elute last. Other tag reporters selectively permeate the pores based on their relative size, and they elute somewhere between the smallest and largest molecules. Size exclusion chromatography is particularly useful for separating tag reporters that are peptides and polypeptides, although this method can be used for other types of tag reporters, including nucleic acid tag reporters.

When selecting a resin for size exclusion chromatography, the chromatographer must match the pore size of the column to the molecular size range of the tag reporters in a sample. Guidelines for selecting appropriate resins for size exclusion chromatography are described, for example, in Wu, C.-S., “Column Handbook for Size Exclusion Chromatography,” Academic Press, San Diego, (1999), as well as in publications provided by chromatography media manufacturers and distributors, such as Vydac, Shodex, Millipore, Pierce and Amersham Biosciences. Exemplary size exclusion chromatography media for separating tag reporters that are nucleic acid molecules diol/dexran resins having a pore size of between 100 and 500 angstroms. Exemplary size exclusion chromatography media for separating tag reporters that are peptides or polypeptides include diol/dextran resins having pore size of between 60 to 100 and 100 to 300 angstroms, respectively. An exemplary size exclusion chromatography media for separating tag reporters that are small compounds, including small peptides having only a few amino acids, is poly(2-hydroxyethyl aspartamide) (Wu, supra (1999). Well-known size exclusion resins that are available in a wide variety of pore sizes suitable for separating tag reporters of various sizes include Sephadex, Separose, Sephacryl, Superose and Superdex.

Separation of tag reporters that are smaller molecules, such as chemical compounds, for example alkylenes and aralkylenes, can be performed using small pore size resins, whereas wide-pore resins generally are used for separating tag reporters that are peptides, polypeptides and nucleic acid molecules.

Separation of nucleic acid tag reporters based on size can also be performed using recently described methods such as matched ion polynucleotide chromatography (MIPC) and slalom chromatography (for example, see U.S. Pat. No. 5,997,742).

Separation of tag reporters based on hydrophobic interactions can be performed by hydrophobic interaction chromatography and closely related reversed-phase chromatography methods. Hydrophobic interaction chromatography (HIC) has generally been most useful for separating small molecules and peptides, while reversed phase chromatography has been more widely applicable to larger molecules, such as polypeptides and nucleic acids. HIC employs a chemically bonded hydrophobic stationary phase, with the mobile phase being more polar than the stationary phase. The basis of HIC is the interaction between hydrophobic parts of tag reporters and a hydrophobic matrix. HIC can be used to separate a variety of types of tag reporters, including organic molecules, oligonucleotides and peptides. Exemplary HIC chromatography media for separating tag reporters that are oligonucleotides, peptides or chemical compounds, include phenyl, butyl or octyl hydrophobic ligands coupled to a sepharose matrix and ether, isopropyl or hydrophobic ligands coupled to a polystyrene/divinylbenzene matrix.

Reverse phase chromatography is a type of chromatography in which the chemically bonded phase is hydrophobic (nonpolar) than the stationary phase. This is “reversed” from normal phase chromatography, in which the stationary phase is hydrophilic (polar), and the starting mobile phase is more nonpolar than the stationary phase. Mobile phase gradients that increase in concentration of an organic modifier (usually acetonitrile or methanol) are commonly used in reverse phase HPLC. These gradients elute solute molecules in order of increasing hydrophobicity. Reverse phase chromatography uses high hydrophobic ligand density and low pH mobile phase with organic modifiers, which tends to denature polypeptides. Thus, the separation is based on the overall hydrophobicity of the polypeptide rather than its surface hydrophobicity.

Various mobile phase additives can be used to provide different selectivity to improve separation of tag reporters. For example, ion pairing reagents may be used in reverse phase HPLC methods. Exemplary ion pairing reagents include trifluoroacetic acid (TFA), which is an anionic ion-pairing reagent, and tetrabutylammonium phosphate, which is a cationic ion pairing reagent.

Reverse phase HPLC can be used to separate a variety of types of tag reporters, including organic molecules, oligonucleotides, peptides and polypeptides. Reversed phase HPLC is particularly useful for separating peptide or polypeptide tag reporters that are closely related to each other. Exemplary reversed phase chromatography media for separating tag reporters that are nucleic acid molecules include C₈/C₁₈ resins having a pore size of 80 to 300 angstroms.

Exemplary reversed phase chromatography media for separating tag reporters that are peptides or polypeptides, include C₈/C₁₈ resins having a pore size of between 60 and 80 angstroms and C₃/C₄/C₈ resins having a pore size of 300 angstroms, respectively. Commercial preparations useful for separating peptides and polypeptides include, for example, Apex WP Octadecyl C₁₈, Octyl C₈, Butyl C₄ and Phenyl, Aquaprep RP-3000 C₄ and C₈, Bakerbond WP Octadecyl C₁₈, Octyl C₈, Butyl C₄ and Diphenyl.

When reverse phase or ion-pair HPLC methods are insufficient to provide adequate separation of all tag reporters, switching to normal phase HPLC may be helpful, because different retention processes provide different selectivity effects. In contrast to the conditions used for reversed phase chromatography, normal phase chromatography involves using a stationary phase is hydrophilic (polar), and the starting mobile phase is more non-polar than the stationary phase. Sample retention is controlled by adsorption to the stationary phase, and molecules must displace solvent molecules from the stationary phase. Normal phase chromatography can be used to separate tag reporters having a variety of physicochemical properties.

Mixed mode chromatography also can be used to separate tag reporters, and is particularly useful for separating oligonucleotide reporter tags. Mixed mode chromatography takes advantage of both hydrophobic and electrostatic interactions between the tag reporters to be separated and the stationary phase. Exemplary mixed mode column packing materials include NACS-12, derivatized aminopropyl silica particles with alkyl and aryl residues.

Tag reporters can be separated based on affinity toward a variety of ligands, such as lectins, dyes, peptides, drugs and other molecules. Lectin affinity chromatography is particularly useful for separating tag reporters that are oligosaccharides, glycosylasparagines and glycopeptides. Dye chromatography can be used to separate a variety of tag reporter types, including nucleic acids and peptides. Affinity chromatography media generally are selected by empirical testing. For example, several different affinity media can be tested to identify the media having the best separation characteristics for a particular set of tag reporters. Many derivatized and activated matices for affinity chromatography, such as those having different reactive side chains, activation chemistries, spacer arm lengths and coupling agents are commercially available, and can be used for preparing a desired affinity media.

A variety of chromatographic media useful for separating tag reporters by size, charge, hydrophobicity and other properties are described, for example, in Snyder et al. “Practical HPLC Method Development,” 2^{nd} ed., New York, John Wiley & Sons (1997).

Selection of a suitable mobile phase for chromatography of tag reporters will depend on the chromatography medium selected and the particular properties of the tag reporters, such as whether the tag reporters are small molecules, nucleic acids, peptides or polypeptides. Exemplary mobile phases for use with ion exchange, size exclusion, reversed phase and hydrophobic interaction chromatography for separating a variety of small molecules and macromolecules are described, for example, in Millner, supra (1999).

Prior to separation by HPLC, a sample can be fractionated or subjected to a pre-separation step, for example, to remove particulate matter or molecules other than reporter tags. In addition to standard biochemical methods for fractionating samples, such as centrifugation, precipitation, filtration and extraction, a variety of HPLC pre-columns or guard columns can be used for this purpose.

Separated tag reporters can be detected using a variety of analytical methods, including detection of intrinsic properties of tag reporters, such as absorbance, fluorescence or electrochemical properties, as well as detection of a detection group or moiety attached to a tag reporter. Although not required, a variety of detection groups or moieties can be attached to tag reporters to facilitate detection after chromatographic separation.

Detection methods for use with liquid chromatography are well known, commercially available, and adaptable to automated and high-throughput sampling. The detection method selected for analysis of tag reporters will depend upon whether the tag reporters contain a detectable group or moiety, the type of detectable group used, and the physicochemical properties of the tag reporter and detectable group, if used. Detection methods based on fluorescence, electrolytic conductivity, refractive index, and evaporative light scattering can be used to detect various types of tag reporters.

A variety of optical detectors can be used to detect a tag reporter separated by liquid chromatography. Methods for detecting nucleic acids, polypeptides, peptides, and other macromolecules and small molecules using ultraviolet (UV)/visible spectroscopic detectors are well known, making UV/visible detection the most widely used detection method for HPLC analysis. Infrared spectrophotometers also can be used to detect macromolecules and small molecules when used with a mobile phase that is a transparent polar liquid.

Variable wavelength and diode-array detectors represent two commercially available types of UV/visible spectrophotometers. A useful feature of some variable wavelength UV detectors is the ability to perform spectroscopic scanning and precise absorbance readings at a variety of wavelengths while the peak is passing through the flowcell. Diode array technology provides the additional advantage of allowing absorbance measurements at two or more wavelengths, which permits the calculation of ratios of such absorbance measurements. Such absorbance rationing at multiple wavelengths is particularly helpful in determining whether a peak represents one or more than one tag reporter.

Fluorescence detectors can also be used to detect fluorescent tag reporters, such as those containing a fluorescent detection group and those that are intrinsically fluorescent. Typically, fluorescence sensitivity is relatively high, providing an advantage over other spectroscopic detection methods when tag reporters contain a fluorophore. Although tag reporters can have detectable intrinsic fluorescence, when a tag reporter contains a suitable fluorescent detection group, it can be possible to detect a single tag reporter in a sample.

Electrochemical detection methods are also useful for detecting tag reporters separated by HPLC. Electrochemical detection is based on the measurement of current resulting from oxidation or reduction reaction of the tag reporters at a suitable electrode. Since the level of current is directly proportional to tag reporter concentration, electrochemical detection can be used quantitatively, if desired.

Evaporative light scattering detection is based on the ability of particles to cause photon scattering when they traverse the path of a polychromatic beam of light. The liquid effluent from an HPLC is first nebulized and the resultant aerosol mist, containing the tag reporters, is directed through a light beam. A signal is generated that is proportional to the amount of the tag reporter present in a sample, and is independent of the presence or absence of detectable groups such as chromophores, fluorophores or electroactive groups. Therefore, the presence of a detection group or moiety on a tag reporter is not required for evaporative light scattering detection.

Mass spectrometry methods also can be used to detect tag reporters separated by HPLC. Mass spectrometers can resolve ions with small mass differences and measure the mass of ions with a high degree of accuracy and sensitivity. Mass spectrometry methods are well known in the art (see Burlingame et al. Anal. Chem. 70:647R-716R (1998); Kinter and Sherman, Protein Sequencing and Identification Using Tandem Mass Spectrometry Wiley-Interscience New York (2000)).

Analysis of data obtained using any detection method, such as spectral deconvolution and quantitative analysis can be manual or computer-assisted, and can be performed using automated methods. A variety of computer programs can be used to determine peak integration, peak area, height and retention time. Such computer programs can be used for convenience to determine the presence of a tag reporter qualitatively or quantitatively. Computer programs for use with HPLC and corresponding detectors are well known to those skilled in the art and generally are provided with commercially available HPLC and detector systems.

The particular tag reporters contained in a sample can be determined, for example, by comparison with a database of known chromatographic properties of reference tag reporters, or by algorithmic methods such as chromatographic pattern matching, which allows the identification of components in a sample without the need to integrate the peaks individually. A computer program useful for chromatographic pattern matching is Millennium³². The identities of tag reporters in a sample can be determined by a combination of methods when large numbers of tag reporters are simultaneously identified, if desired.

A variety of commercially available systems are well-suited for high throughput analysis of tag reporters. Those skilled in the art can determine appropriate equipment, such as automated sample preparation systems and autoinjection systems, useful for automating HPLC analysis of tag reporters. Automated methods can be used for high-throughput analysis of tag reporters, for example, when a large number of samples are being processes or for multiplexed application of the methods of the invention for detecting target analytes.

Those skilled in the art will be aware of quality control measures useful for obtaining reliable analysis of tag reporters, particular when analysis is performed in a high-throughput format. Such quality control measures include the use of external and internal reference standards, analysis of chromatograph peak shape, assessment of instrument performance, validation of the experimental method, for example, by determining a range of linearity, recovery of sample, solution stability of sample, and accuracy of measurement.

The invention provides methods for detecting a variety of target analytes including nucleic acids, and polypeptides such as specific binding pairs of polypeptides. Several different combinations of cleavable linkages and cleavage-inducing moieties, for example nucleases or visible light, can be utilized in the invention. In addition, the invention provides methods for detecting a variety of target analytes using tagged probes with various configurations. For example, a tagged probe can contain a cleavage-inducing moiety directly attached to the tagged probe. In addition, for example, a cleavage-inducing moiety and cleavable tag reporter can be on separate reagents that are brought into proximity to each other resulting in release of a tag reporter.

The invention provides a method for detecting a target nucleic acid sequence by: (a) contacting one or more target nucleic acid sequences with a set of tagged probes under conditions sufficient for hybridization of a target nucleic acid sequence with a tagged probe, where the tagged probes contain a mobility modifier attached to a nucleic acid target binding moiety by a bond that is cleavable by a nuclease, and where the nucleic acid target binding moiety contains at least one bond that is resistant to the nuclease; (b) treating the tagged probe hybridized to the target nucleic acid with a nuclease under conditions sufficient for cleavage of the nuclease-cleavable bond to release a tag reporter; (c) separating said tag reporter using a chromatographic method; and (d) detecting a tag reporter corresponding to a known target sequence.

The method can further include an additional step of separating one or more cleaved tagged probes from un-cleaved or partially-cleaved tagged probes. Separation can be accomplished using capture ligands, such as biotin or other affinity ligands, and capture agents, such as avidin, streptavidin, an antibody, a receptor, or a functional fragment thereof, having specific binding activity to the capture ligand. A tagged probe, or a target-binding moiety of a tagged probe, can contain a capture ligand having specific binding activity for a capture agent. For example, the target-binding moiety of a tagged probe can be biotinylated or attached to an affinity ligand using methods well known in the art. After the tag reporter is cleaved from the tagged probe, the remaining part of the tagged probe with the target-binding moiety and biotin can be removed by, for example, strepavidin agarose beads. A capture ligand and capture agent can also be used to add mass to the remaining part of the tagged probe such that it can be excluded from the mass range of the tag reporters separated by chromatography.

A nuclease can also cleave other bonds in the target-binding moiety or target nucleic acid that are nuclease-susceptible. However, an advantage of having at least one nuclease-resistant bond in the target-binding moiety is that a tagged probe will yield a single sized species of released tag reporter upon cleavage. Nuclease-cleavable bonds can include, for example, a phosphodiester bond, and nuclease-resistant bonds can include, for example, thiophosphate, phosphinate, phosphoramidate, or a linker other than a phosphorous acid derivative, such as amide and boronate linkages.

Several nucleases are known in the art that can be used to cleave different types of nucleic acids. For example, nucleases are available that can cleave double-stranded DNA, for example, DNAse I and Exonuclease III, or single-stranded DNA, for example, nuclease S1. Nucleases include enzymes that function solely as nucleases as well as multi-functional enzymes that contain nuclease activity such as, for example, DNA polymerases like Taq polymerase that have 5′ nuclease activity. Several derivatives of Taq polymerases derived from different bacterial species or from designed mutations are known which cleave specific structures of nucleic acid hybrids (Kaiser et al., J. Biol. Chem. 274:21387-21394 (1999); Lyamichev et al., Proc. Natl. Acad. Sci. USA 96:6143-6148 (1999); Ma et al., J. Biol. Chem. 275:24693-24700 (2000)). For example, Cleavase™ enzymes (Third Wave Technologies) have been developed that cleave only at specific nucleic acid structures.

A target nucleic acid used in the methods of the invention can include any nucleic acid that can be bound by a tagged probe. For example, RNA or single-stranded or double-strand DNA. In one embodiment, the target nucleic acid can be a single nucleotide polymorphism (SNP).

For detecting SNPs, various techniques can be employed of varying complexity. In one embodiment, a primer can be employed that terminates at the nucleotide immediately preceding the SNP. The tag reporter can be bound to the primer and a ligand can be bound to the nucleotide reciprocal to the SNP. In one approach, four vessels can be used, each with a different labeled nucleotide, for example, each nucleotide can have, or be made to have, different masses in a mass spectrometer. In another approach, one vessel can be employed with each of the labeled nucleotides having a different mass modifier. The primers can be extended and then captured, for example, by having an affinity ligand, such as biotin attached to the nucleotide, and contacting the extension mixture with the reciprocal receptor, such as streptavidin, bound to a support. The tag reporter can then released by, for example, a nuclease and analyzed. By grouping targets of interest having the same nucleotide for a SNP, the assay can be multiplexed for a plurality of targets. Other methods include having probes where the SNP is mismatched. The mismatching nucleotide is labeled with the tag reporter. When the SNP is present, the tag reporter labeled nucleotide will be released for detection, for example, by mass spectrometry. See U.S. Pat. No. 5,811,239.

Each SNP detection sequence can have at least one nucleotide modified with a tagged probe, which can be detected, for example, by mass spectrometry. Usually, the modified nucleotide will be at the 5′ end of the sequence, but the modified nucleotide can be anywhere in the sequence, particularly where there is a single nuclease susceptible linkage in the detection sequence. Since the determination is based on at least partial degradation of the SNP detector sequence, having the modified nucleotide at the end ensures that if degradation occurs, the tag reporter will be released. Since nucleases can cleave at other than the terminal phosphate link, it is desirable to prevent cleavage at other than the terminal phosphate link. In this way one avoids the confusion of having the same tag reporter joined to different numbers of nucleotides after cleavage. Therefore, specific signal to noise can be increased using nuclease resistant bonds at positions distal to the cleavable linkage. Cleavage at the terminal phosphate can be relatively assured by using a linker that is not cleaved by the nuclease, more particularly having only the ultimate linkage susceptible to hydrolysis by a nuclease. If desired, all of the linkers other than the ultimate linker can be resistant to nuclease hydrolysis.

A plurality of SNPs or other polymorphisms can be simultaneously determined by combining target DNA with a plurality of reagent pairs under conditions of primer extension. Each pair of reagents includes a primer which binds to target DNA and a SNP detection sequence, normally labeled, which binds to the site of the SNP and has a tag, usually at its 5′ end and the base complementary to the SNP, usually at other than a terminus of the SNP detection sequence. The conditions of primer extension can employ a polymerase having 5′-3′ exonuclease activity, dNTPs and auxiliary reagents to permit efficient primer extension. The primer extension is performed, whereby detector sequences bound to the target DNA are degraded with release of the tag. By having each SNP associated with its own tag, one can determine the SNPs which are present in the target DNA for which pairs of reagents have been provided. In one SNP determination protocol, the primer includes the complementary base of the SNP. This protocol is referred to as Invader™ technology, and is described in U.S. Pat. No. 6,001,567.

In another embodiment, a plurality of oligonucleotide probes or a target polynucleotide sample can be bound to a surface of a solid support such as an array. Arrays can be convenient for handling a large number of nucleic acid probes when performing multiplex assays. Methods for constructing arrays are well known in the art. See, for example, U.S. Pat. No. 5,744,305 (Fodor, et al.); PCT application WO 89/10977; Gamble, et al., WO97/44134; Gamble, et al., WO98/10858; Baldeschwieler, et al., WO95/25116; Brown, et al., U.S. Pat. No. 5,807,522; and the like.

Another embodiment of the invention utilizes a cleavage-inducing moiety that is physically attached to the tagged probe. For example, a tagged probe can contain a tag reporter region attached to a target-binding moiety by a bond that is cleavable when the attached cleavage-inducing moiety is activated. An advantage to having the cleavage-inducing moiety attached to the tagged probe is that the cleavage agent is produced locally and in a one-to-one correspondence to tag reporter. This arrangement, and other close proximal arrangements as described further below, can facilitate both an increase in specific signal and a decrease in non-specific background or noise. The proximity of the cleavage-inducing moiety to the cleavable linker increases the likelihood of cleavage, thus increasing the signal. A further advantage to having the cleavage-inducing moiety attached or in close proximity to the cleavable linker is that this moiety is less likely to be involved in non-specific cleavage reactions. Therefore, proximal arrangements of the cleavage-inducing moiety to the cleavable linker lead to a better signal-to-noise ratio in the assay.

Another advantage to proximal arrangements of the cleavage-inducing moiety to the cleavable linker can be a reduction in undesirable side reactions if the cleavage agent is, for example, toxic, volatile, or highly reactive. Another way to avoid undesirable side reactions is to use a gentle cleavage agent, for example, visible light. This type of cleavage can be advantageous when assaying biomolecules such as nucleic acids and proteins which can be damaged by reagents such as ultra-violet light, strong acids or bases, but are stable in the presence of visible light.

The invention provides a method for detecting a target analyte by contacting a target analyte with a set of tagged probes attached to a cleavage-inducing moiety under conditions sufficient for binding of the analyte with a tagged probe, where the tagged probes contain a mobility modifier attached to a target binding moiety by a cleavable linkage and where the cleavable linkage is susceptible to cleavage when the cleavage-inducing moiety is activated by visible light; separating tagged probes bound to a target binding moiety from unbound tagged probes; activating the cleavage-inducing moiety with visible light to release a tag reporter; and detecting a chromatographic property of the tag reporter, where the chromatographic property uniquely corresponds to a known target analyte.

Separation of tagged probes bound to a target binding moiety from unbound tagged probes can be performed using a variety of methods. For example, when an analyte is immobilized, unbound tagged probes can be separated by a washing step. An analyte can be immobilized to a solid surface, a bead or another matrix, using well-known methods. When an analyte is in solution, separation of unbound tagged probes can be achieved by well-known analytical methods, such as electrophoresis or chromatography.

The method can further include an additional step to separate one or more cleaved tagged probes from un-cleaved or partially-cleaved tagged probes using capture ligands and capture agents having specific binding activity to the capture ligand.

In addition, the method can be used in a multiplex format when one or more target analytes further comprise a plurality of different target analytes. As described above, target analytes can be polypeptides, proteins, peptides, polysaccharides, nucleic acids, and small molecules. Therefore, the target binding moiety can be a ligand, antiligand, receptor, antibody, biotin, avidin, strepavidin, protein A and polynucleotide, or a functional fragment thereof, that binds to the target analyte.

One of the advantages of the methods of the invention is the ability to perform multiplex assays. In multiplex assays several analytes can be detected simultaneously. In a multiplex format, sets of tagged probes are used such that the resulting tag reporter has a unique characteristic, for example a unique mass charge-to-mass ratio or chromatographic property, that differentiates the tag reporter from other tag reporters in the same set. A multiplex experiment can be used to detect 2 or more analytes, 10 or more analytes, 100 or more analytes, 1,000 or more analytes, or 10,000 or more analytes in the same assay. The number of tagged probes used in a multiplex assay is equal to or greater than the number of analytes to be detected. For example, when a multiplex experiment is used to detect 100 analytes, 100 or more tagged probes that result in 100 or more tag reporters having unique properties are used. The number of analytes that can be detected in a single assay is limited only by the number of distinct tag reporters that can be detected in a single assay. HPLC methods can resolve small differences in mass, charge-to-mass ratio and other properties of tag reporters allowing the detection of a large number of tag reporters in a single assay.

As described previously, tagged reporters can contain a cleavage-inducing moiety. The cleavage-inducing moiety can further comprise a photosensitizer or a chemi-activated sensitizer. For example, the cleavage-inducing moiety can be a sensitizer capable of generating singlet oxygen and the cleavable linkage can be susceptible to cleavage by singlet oxygen. In addition, the cleavage-inducing moiety can be a sensitizer such as a benzophenone, 9-thioxanthone, eosin, 9,10,-dibromoanthraene, methylene blue, metallo-porphyrin, chloroperoxidase or myeloperoxidase. Furthermore, the cleavage-inducing moiety can comprise two or more cleavage-inducing moieties.

In one embodiment, the cleavage-inducing moiety acts in such a manner as to produce an active short-lived species that is able to act upon the cleavable linkage and release the releasable portion only when the two reagents are brought into close proximity in relation to the presence of the analyte. A short-lived species is advantageous to limit undesirable side reactions when the species is toxic, volatile, or highly reactive and to limit non-specific reactions thus reducing background noise in the assay. In one embodiment of the present invention the first reagent is a sensitizer reagent capable of generating singlet oxygen and the second reagent comprises a portion releasable by the generated singlet oxygen. Singlet oxygen is a short-lived agent and so has the advantages of a short-lived agent as described above. Under the circumstance of the close proximity of the two reagents in relation to the presence of the analyte, the short-lived species is able to cleave the cleavable linkage.

In another embodiment of the invention, a feature involves bringing into close proximity, in relation to the presence of the target analyte, a first reagent that contains a cleavage-inducing moiety and a second reagent that contains a portion that is releasable by the action of the cleavage-inducing moiety. The reagents are brought into close proximity in relation to the presence of the analyte by virtue of some interaction or binding event involving the analyte. The releasable portion is released upon activation of the cleavage-inducing moiety when the analyte is present in the sample and influences the extent that the above reagents are brought into close proximity. This close proximal relationship is advantageous in that it results in an increase in specific signal and a decrease in non-specific signal thus improving the signal-to-noise ratio, as described previously. In addition, since the cleavage agent is produced locally, if the cleavage agent is toxic or reactive this arrangement can limit the chance of undesirable side reactions.

The methods described previously for detecting a target nucleic acid also can be performed using a cleavage-inducing moiety other than a nuclease. For example, the determination of a target nucleic acid can be performed using two oligonucleotide probes, each binding to different regions of the target polynucleotide. One of the oligonucleotide probes can be labeled with a cleavage-inducing moiety such as a sensitizer and the other oligonucleotide probe can be a tagged probe. The oligonucleotide probes can be selected so that they bind to regions of the target nucleic acid that permit the cleavage-inducing moiety and the cleavable linkage to be brought into proximity when the target nucleic acid is hybridized. Upon binding of all three components and activation of the cleavage-inducing moiety, the cleavable linkage is cleaved releasing the tagged reporter moiety of the tagged probe.

The cleavage-inducing moiety and the tagged moiety may be linked to their respective oligonucleotide probes at the 3′-end or the 5′-end or at any point that is feasible along the nucleotide chain. One consideration is that the cleavage-inducing moiety and the cleavable linkage be brought into sufficient proximity upon hybridization to the target nucleic acid that the cleavable linkage can be cleaved. In one approach the 3′-end of one oligonucleotide probe is labeled with either the cleavage-inducing moiety or a tagged moiety and the 5′-end of the other oligonucleotide probe is labeled with the other of the above moieties. In this approach the oligonucleotide probes are designed so that the binding to the target nucleic acid brings the labeled ends internal to the duplexes formed. In other words the 3′-end labeled oligonucleotide probe binds downstream on the target sequence from the region to which the 5′-end labeled oligonucleotide probe binds.

Another embodiment for detection of a target nucleic acid employs three oligonucleotide probes, each binding to different regions of the target polynucleotide. A first oligonucleotide probe is labeled with an activator moiety that is capable of generating a reaction product, which in turn is able to activate a cleavage-inducing moiety, such as a sensitizer, incorporated in a second oligonucleotide probe. The cleavage-inducing moiety, once activated, is then capable of acting on the third oligonucleotide probe containing a cleavably-linked tagged moiety, causing release of a tagged reporter. As with the two oligonucleotide approach described above, the three oligonucleotide probes should bind to the target sequence in a manner that brings the activator moiety, cleavage-inducing moiety, and tagged moiety into sufficient proximity that the cleavable linkage can be cleaved upon activation. One specific embodiment of this approach would have the three oligonucleotide probes bound adjacently to one another on the target sequence to be detected.

The aforementioned method can be employed to detect multiple target polynucleotides simultaneously by utilizing appropriate sets of oligonucleotide probes and appropriate tagged moieties that permit separation and detection of the released reporter groups, with concomitant identification of the respective target nucleic acids. The methods of the invention are particularly suited for analysis of complex mixtures of target nucleic acids employing array technology and microfluidics.

In a particular embodiment of the above method, the cleavage-inducing moiety is able to intercalate into the nucleic acid duplex created when the oligonucleotide probe binds to a respective target polynucleotide. In this regard the cleavage-inducing moiety can be attached to one of the oligonucleotide probes or it may be a separate reagent. In the latter embodiment a single oligonucleotide probe comprising a tagged moiety can be used for each target nucleic acid.

The methods for detecting a target nucleic acid sequence using an oligonucleotide probe containing a cleavage-inducing moiety and an oligonucleotide containing a cleavably-linked tagged moiety can also be used to detect a target analyte, such as a polypeptide.

The invention provides a method for detecting a target analyte by: (a) contacting a target analyte with a set of first and second binding reagents under conditions sufficient for binding of a target analyte with the first and second binding reagents, where each of the first binding reagents contains a cleavage-inducing moiety and a target binding moiety, and each of the second binding reagents contains a tagged probe having a mobility modifier attached to a target binding moiety by a cleavable linkage, and where the cleavable linkage is susceptible to cleavage when in proximity to an activated cleavage-inducing moiety; (b) activating the cleavage-inducing moiety to release a tag reporter; (c) and separating said tag reporter using a chromatographic method, and (d) detecting a tag reporter corresponding to target analyte.

An additional step that can be added to the method described above is to separate one or more of the cleaved tagged probes from un-cleaved or partially-cleaved tagged probes using capture ligands and capture agents having specific binding affinity to the capture ligand.

The first binding reagent comprises a cleavage-inducing moiety. The cleavage-inducing moiety can further comprise a photosensitizer or a chemi-activated sensitizer.

Assays can be performed in a competitive mode or a sandwich mode. In an example of a competitive mode, the target competes with a labeled binding member for the reciprocal member. In this mode, the binding sites of the reciprocal binding member become at least partially filled by the target, reducing the number of available binding sites for the labeled reciprocal binding member. Thus, the number of labeled binding members that bind to the reciprocal binding member will be in direct proportion to the number of target molecules present. In a sandwich mode, the target is able to bind at the same time to different binding members, that is, a first member and a second member that binds at a site of the target molecule different from the site at which the first member binds. The resulting complex has three components, where the target serves to link the first and second members.

The methodologies that can be employed can be competitive or non-competitive, heterogeneous or homogeneous. Heterogeneous techniques normally involve a separation step, where unbound label is separated from bound label. On the other hand, homogeneous assays do not require, but can employ, a separation step. Non-competitive assays are usually sandwich assays involving the binding of an analyte to two target binding moieties specific for the analyte whereas competitive assays usually involve competition for binding sites between an analyte and an analyte analog.

In addition, in many heterogeneous assays it can be required that the unbound labeled reagent be separable from the bound labeled reagent. This can be achieved in a variety of ways, each requiring a reagent bound to a solid support that distinguishes between the complex of labeled reagent and target. The solid support can have the complex directly or indirectly bound to the support for directly bound, one can have the binding member or tagged probe covalently or non-covalently bound to the support. The solid support can be a vessel wall, for example, microtiter well plate well, capillary, plate, slide, beads, including magnetic beads, liposomes, or the like. The primary characteristics of the solid support is that it permits segregation of the bound labeled specific binding member from unbound probe and that the support does not interfere with the formation of the binding complex, nor the other operations of the determination.

One example of an assay is a sandwich-type immunoassay, which allows for the qualification and quantification of known antigens. In this assay, a matched pair of antibodies forms a sandwich with an antigen bringing the two antibodies in close proximity. One of these antibodies can be conjugated with one or more tag moieties to form a tagged probe. The tag moiety can be linked to an antibody by a singlet oxygen labile linkage, which allows the release of a tag reporter after reaction with singlet oxygen. The second antibody can be conjugated, for example, to a sensitizer dye that produces singlet oxygen when irradiated. When the two antibodies form a sandwich, the singlet oxygen cleaves the cleavable linkage to release a tag reporter. The tag reporter is separately detectable by virtue of, for example, its unique mass or chromatographic property. Detection of the reporter is related to the presence of the antigen. In addition, detection of the reporter is related to the amount of the antigen.

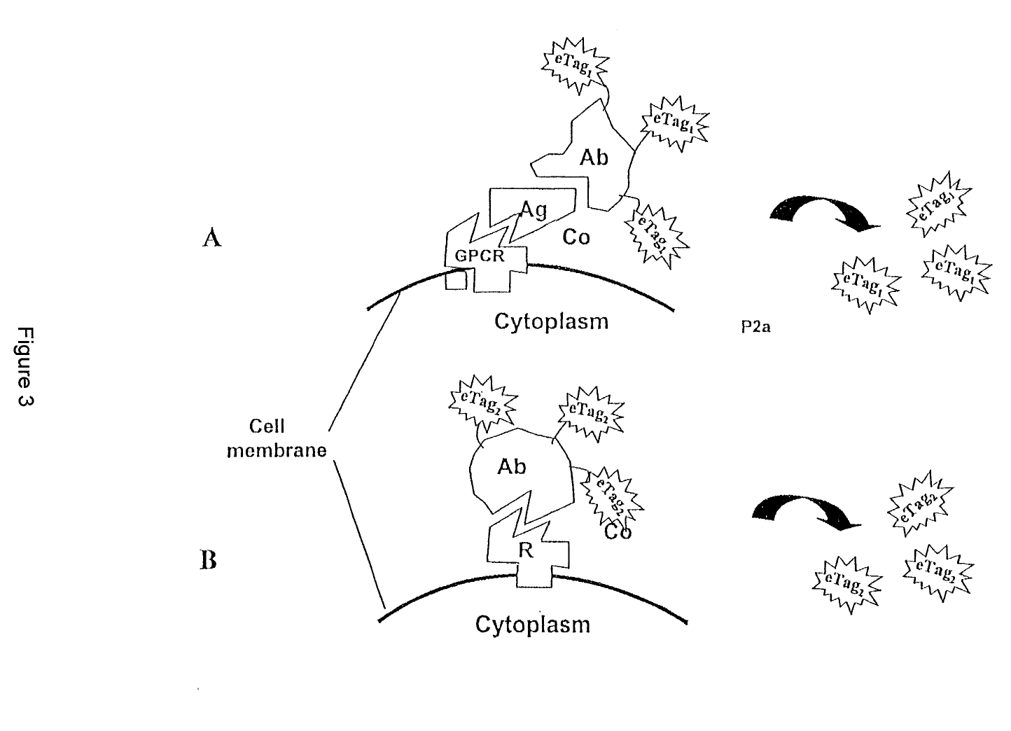

One particular embodiment of a method of use in accordance with the present invention is a multiplexed quantitation of cell surface receptors. Referring to FIG. 3A, a cell membrane is shown exhibiting a GPCR receptor present on the cell membrane. Co represents a cofactor for the binding of a protein antigen to the GPCR receptor. The tagged probe reagent is depicted as an antibody (Ab) with several tag moieties releaseably linked thereto. Another reagent is the cleavage-inducing reagent, which can be bound to the surface of the cell membrane by a component that specifically binds to a component of the cell surface that is not the subject of the method, for example, a generic receptor. Alternatively, the cleavage-inducing reagent can be incorporated into the cell membrane, as will described further below. When the receptor is present, the protein antigen binds to it and then the Ab binds to the antigen, bringing the releasable tag moiety in close proximity to the cleavage-inducing reagent. After cleavage, the tag reporters are released, detected and quantitated, and related to the amount of the receptor present. This embodiment can be employed to screen numerous proteins for their ability to interact with the receptor on the cell surface.

A variation of the above is depicted in FIG. 3B. The receptor on the cell surface is represented by R. As in FIG. 3A, the tagged probe reagent is depicted as an antibody (Ab) with several tag moieties cleavably linked thereto. Another reagent is the releasing inducing reagent as discussed above. When the receptor is present, Ab binds to the receptor bringing the releasable tag moiety in close proximity to the release-inducing reagent. The tag reporters are released, detected and quantitated and related to the amount of the receptor present. This embodiment can be employed to screen numerous cell lysates for the presence of the receptor of interest. Numerous antibody reagents can be employed to screen a cell lysate for the presence of proteins of interest in a single assay.

In addition to identifying a target analyte or analytes, this embodiment also can be used to determine whether two target-binding moieties are able to bind to the same analyte. For example, this method can be used to determine whether two target-binding moieties, such as two antibodies, are able to bind to the same analyte or antigen. The production of a tag reporter would indicate that the two reagents were able to bind the same target thus bringing the reagent with the cleavage-inducing moiety and the reagent with the cleavable linkage into close proximity.

The methods of the invention offer a high degree of versatility for screening unknown materials. The unknown entity can be the target-binding moiety of the tagged probe or of the cleavage-inducing reagent. On the other hand, the unknown entity can be the analyte that can be bound by the target-binding moiety of the tagged probe or the cleavage-inducing reagent or both. Thus, as can be seen, known and unknown entities can be selectively chosen for the reagents and the analyte by the skilled artisan to accommodate a broad range of potential assays and needs.

The invention also provides a method for identifying a binding partner of a specific binding pair by: (a) incorporating a cleavage-inducing moiety into a first binding partner of a specific binding pair; (b) contacting the first binding partner having an incorporated cleavage-inducing moiety with a set of second binding partners under conditions sufficient for binding, where each of the second binding partners contains a tagged probe having a mobility modifier attached to a target binding moiety by a cleavable linkage, where the cleavable linkage is susceptible to cleavage when in proximity to an activated cleavage-inducing moiety; (c) activating the cleavage-inducing moiety to release a tag reporter; (d) separating said tag reporter using a chromatographic method, and (e) detecting a tag reporter corresponding to a known second binding partner of a specific binding pair. The additional step of separating one or more of the cleaved tagged probes from un-cleaved or partially cleaved tagged probes using capture ligands and agents can also be performed as described above. The chromatographic property of the tag reporter can be determined, for example, using size exclusion, ion exchange or reversed phase chromatography.

Although the invention has been described above with reference to binding reagents, any of the previously described formats or modes can also be performed by directly incorporating the tagged probe, the cleavage-inducing moiety or both into binding pairs. For example, with protein binding pairs, one protein of a binding pair can be bound by a first reagent that contains a cleavage-inducing moiety and the second protein of a binding pair can be bound by a second reagent that contains a portion that is releasable by the action of the cleavage-inducing moiety. If the two proteins interact, they will be brought into close proximity and a tag reporter will be cleaved and released. A similar method is provided by the invention to screen for a binding partner of a specific binding pair. In this method a cleavage-inducing moiety is incorporated into a first binding partner of a specific binding pair. The first binding partner with the cleavage-inducing moiety is contacted with a set of potential second binding partners that contain a tagged probe with a releasable portion. If two binding partners interact, a tag reporter is cleaved off and released for detection. The unique physical properties of the tag reporter identify the second binding partner.

A first binding partner, or set of first binding partners, containing a cleavage inducing moiety can be prepared by incorporating a cleavage inducing moiety into the binding partner by covalent or non-covalent linkage. The method selected for incorporating a cleavage inducing moiety into a first binding partner will vary depending on the nature of the particular binding partner selected, and whether the cleavage inducing moiety will be incorporated during synthesis or post-synthetically.

A first binding partner that is a polypeptide can be incorporated with a cleavage inducing moiety by common modification chemistries such as esterification or amidation at a carboxyl group such as a glutamic acid or aspartic acid residue or carboxyl terminus of a peptide or polypeptide; alkylation or acylation of a histidine residue; alklation, acylation or oxidation at a thiol such at a cysteine residue; alkylation, acylation or oxidation at a thioester such as a methionine residue; alkylation or acylation at an amino group such as a lysine or amino terminus; nitration, oxidation, diazotization of a phenol such as a tyrosine residue; acylation at a hydroxy such as a serine or threonine residue; condensation with diones at a guanidino such as an arginine residue; oxidation of an indole such as a tryptophan residue; and oxidation of glycans. When there are no suitable amino acid side chains for modification, reagents such as photoactivated reagents and cross-linking reagents can be employed. Affinity labeling reagents also can be employed for incorporating a cleavage-inducing moiety into a polypeptide, particularly when labeling a group of related polypeptides having a common epitope recognized by an affinity reagent.

A first binding partner that is a polynucleotide can be incorporated with a cleavage inducing moiety using various well-known 5′ or 3′ modification methods, such as coupling of a reagent to a 5′ hydroxyl of an oligonucleotide. For convenience, labeling reagents can be compatible with automated DAN synthesizers. A variety of well-known chemistries can be used to incorporate a cleavage inducing moiety into an organic compound, lipid, carbohydrate, or other molecule.

Indirect attachment of a cleavage-inducing moiety onto a first binding partner, or set of first binding partners, can be performed by non-covalent binding between the first binding partner and a selective agent, such as an antibody, receptor, ligand, native polypeptide subunit, anti-sense polynucleic acid, or other type of target binding moiety.

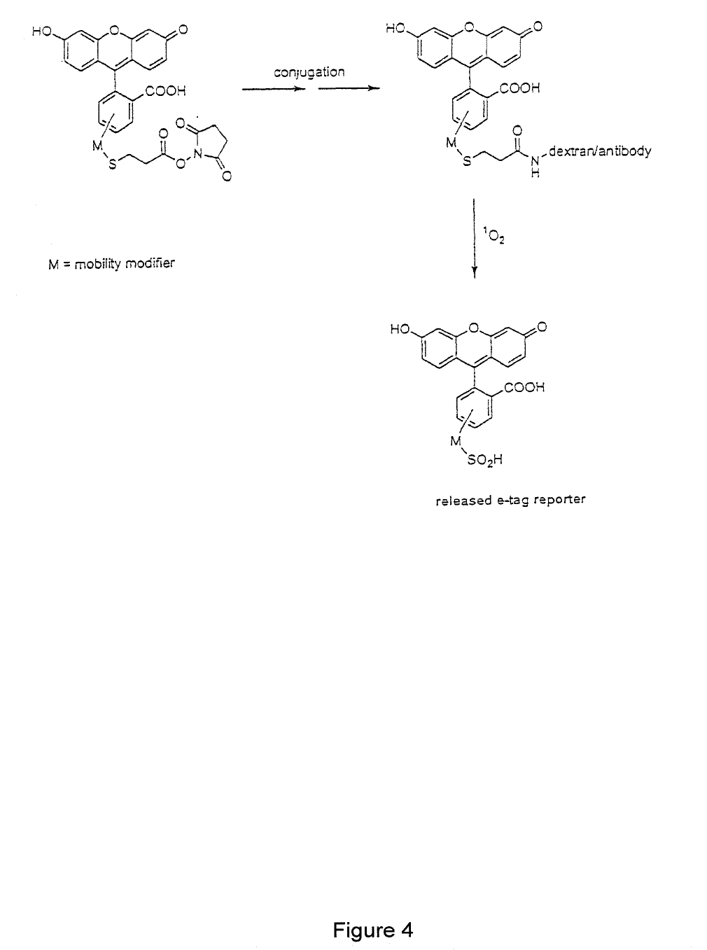

A second binding partner can be prepared by incorporating a tag moiety or e-tag moiety directly or indirectly into the binding partner. As described herein, it is understood that a cleavable linkage exists between a tag moiety and a second binding. A tag moiety can be incorporated into a second binding partner directly, for example, during chemical synthesis of one or a set of second binding partners. Methods for synthetically incorporating a tag moiety into a binding partner, including methods for combinatorial synthesis of sets of binding partners, are described below. FIG. 4 depicts a method for cojugating a tag moiety to an antibody to prepare a tagged probe. Also shown is the reaction of the resulting probe with singlet oxygen to cleave the tag reporter, which can then be separated by HPLC and detected.

A tag moiety can be attached to a second binding partner indirectly, for example, by binding a tagged probe or e-tag probe to a molecule, such as an antibody or receptor, that binds to the second binding partner.

Incorporation of cleavage-inducing moieties or tagged probes into binding pairs can also be used in a multiplex format where the first binding partner contains a plurality of different first binding partners. These different first binding partners can contain distinctive cleavage-inducing moieties. The first binding partner can be, for example, a ligand, antiligand, nucleic acid, or a functional fragment thereof and can contain polypeptides, proteins, peptides, polysaccharides, nucleic acids, and small molecules. In addition, the second binding partner can be a ligand, antiligand, nucleic acid, or a functional fragment thereof. Furthermore, the second binding partner can contain a target binding moiety, for example, a moiety that specifically binds to the first binding partner. This target binding moiety can be a ligand, antiligand, receptor, antibody, biotin, avidin, strepavidin, protein A and polynucleotide, or a functional fragment thereof.

In one embodiment, the methods of the invention can be used to screen for ligands for receptors, for example, to identify ligands for orphan G-protein coupled receptors. There are a large number of specific binding pairs associated with receptors, such as polyclonal and monoclonal antibodies, enzymes, surface membrane receptors, lectins, and ligands for the receptors, which can be naturally occurring or synthetic molecules, protein or non-protein such as drugs, hormones, and enzymes.

In this embodiment, the first binding partner has an incorporated cleavage-inducing moiety. The cleavage-inducing moiety can further comprise a photosensitizer or a chemi-activated sensitizer. For example, the cleavage-inducing moiety can be a sensitizer capable of generating singlet oxygen and the cleavable linkage can be susceptible to cleavage by singlet oxygen. In addition, the cleavage-inducing moiety can be a sensitizer such as benzophenone, 9-thioxanthone, eosin, 9,10,-dibromoanthraene, methylene blue, metallo-porphyrin, chloroperoxidase or myeloperoxidase. Furthermore, the cleavage-inducing moiety can further comprise two or more cleavage-inducing moieties.

It is understood that modifications which do not substantially affect the activity of the various embodiments of this invention are also included within the definition of the invention provided herein. Accordingly, the following examples are intended to illustrate but not limit the present invention.

### EXAMPLE I

### Detection of Multiple Tag Reporters Using HPLC

This example shows methods for preparing tag reagents and for performing a multiplexed assay for detecting multiple analytes.

A. Synthesis of Tag Reagents

Conjugation of Sensitizer Molecules to Assay Reagents

Sensitizer molecules can be conjugated to an antibody, antigen, avidin, biotin, mononucleotides, polynucleotides, small molecules, large molecules and others by various methods and configurations. For example, an activated (NHS ester, aldehyde, sulfonyl chloride, etc) sensitizer (Rose Bengal, phthalocyanine, etc.) can be reacted with reactive amino-group containing moieties (antibody, avidin or other proteins, H2N-LC-Biotin, aminodextran, amino-group containing other small and large molecules). The formed conjugates can be used directly (for example the antibody-sensitizer conjugate, Biotin-LC-sensitizer, etc.) in various assays. Also, the formed conjugates can be further coupled with antibody (for example, aminodextran-sensitizer conjugate containing 20-200 sensitizers and 200-500 amino-groups can be coupled to periodate oxidized antibody molecules to generate the antibody-dextran-sensitizer conjugate) or with the antibody and a particle. For example, aminodextran-sensitizer conjugate containing 20-200 sensitizers and 200-500 amino-groups can be coupled to carboxylated polystyrene beads by EDC coupling chemistry to form the sensitizer-aminodextran-particle conjugate. Methods for incorporation of a sensitizer into a particle are given in, e.g., U.S. Pat. No. 5,340,716. Then the Na-periodate oxidized antibody molecules can be reacted with the amino-groups of the aminodextran molecule, in presence of sodium cyanoborohydride, to generate the antibody-dextran-sensitizer-particle conjugate). It should be noted that instead of an antibody molecule, avidin or other molecules can be used.

Preparation of Pro2, Pro4, and Pro6 through Pro13

Pro2, Pro4, Pro6, Pro7, Pro8, Pro9, Pro10, Pro11, Pro12, and Pro13 are carboxyfluorescein-derived tag moieties. The first step involves the reaction of a 5- or 6-FAM with N-hydroxysuccinimide (NHS) and 1,3-dicylcohexylcarbodiimide (DCC) in DMF to give the corresponding ester, which was then treated with a variety of diamines to yield the desired amide, compound 1. Treatment of compound 1 with N-succinimidyl iodoacetate provided the expected iodoacetamide derivative, which was not isolated but was further reacted with 3-mercaptopropionic acid in the presence of triethylamine. Finally, the resulting β-thioacid (compound 2) was converted, as described above, to its NHS ester. The various tag moieties were synthesized starting with 5- or 6-FAM, and one of various diamines (H2N ˆ X ˆ NH2). The radioisomer of FAM and the chemical entity of “X” within the diamine are indicated in the table below for each of the tag moieties synthesized.

| tag moiety | FAM | X |
|---|---|---|
| Pro2 | 5-FAM | C(CH3)2 |
| Pro4 | 5-FAM | no carbon |
| Pro6 | 5-FAM | (CH2) 8 |
| Pro7 | 5-FAM | CH2OCH2CH2OCH2 |
| Pro8 | 5-FAM | CH2CH2OCH2CH2OCH2CH2OCH2CH2 |
| Pro9 | 5-FAM | 1,4-phenyl |
| Pro10 | 6-FAM | C(CH3)2 |
| Pro11 | 6-FAM | no carbon |
| Pro12 | 6-FAM | CH2OCH2CH2OCH2 |
| Pro13 | 6-FAM | CH2CH2OCH2CH2OCH2CH2OCH2CH2 |

Synthesis of Compound 1

To a stirred solution of 5- or 6-carboxyflourescein (0.5 mmol) in dry DMF (5 mL) were added N-hydroxysuccinimide (1.1 equiv.) and 1,3-dicylcohexylcarbodiimide (1.1 equiv.). After about 10 minutes, a white solid (dicyclohexylurea) started forming. The reaction mixture was stirred under nitrogen at room temperature overnight. TLC (9:1 CH2Cl2-MeOH) indicated complete disappearance of the starting material. The supernatant from the above mixture was added dropwise to a stirred solution of diamine (2-5 equiv.) in DMF (10 mL). As evident from TLC (40:9:1 CH2Cl2-MeOH—H2O), the reaction was complete instantaneously. The solvent was removed under reduced pressure. Flash chromatography of the resulting residue on Iatrobeads silica provided the desired amine (compound 1) in 58-89% yield. The 1H NMR (300 MHz, DMSO-d6) of compound 1 was in agreement with the assigned structure.

Synthesis of Compound 2

To the amine (compound 1) (0.3 mmol) were sequentially added dry DMF (10 mL) and N-succinimidyl iodoacetate (1.1 equiv.). The resulting mixture was stirred at room temperature until a clear solution was obtained. TLC (40:9:1 CH2Cl2-MeOH—H2O) revealed completion of the reaction. The above reaction solution was then treated with triethylamine (1.2 equiv.) and 3-mercaptopropionic acid (3.2 equiv.). The mixture was stirred at room temperature overnight. Removal of the solvent under reduced pressure followed by flash chromatography afforded the β-thioacid (compound 2) in 62-91% yield. The structure of compound 2 was assigned on the basis of its 1NMR (300 MHz, DMSO-d6).

Synthesis of Pro2, Pro4, and Pro6 Through Pro13

To a stirred solution of the β-thioacid (compound 2) (0.05 mmol) in dry DMF (2 mL) were added N-hydroxysuccinimide (1.5 equiv.) and 1,3-dicylcohexylcarbodiimide (1.5 equiv.). The mixture was stirred at room temperature under nitrogen for 24-48 h (until all of the starting material had reacted). The reaction mixture was concentrated under reduced pressure and then purified by flash chromatography to give the target molecule in 41-92% yield.

B. Tag Reporter Assay for Protein Analysis

Direct Conjugation of Tag Moieties to Antibodies

Tag moieties were synthesized with an NHS ester end that reacted with primary amines of the antibody to form a stable amide linkage. This resulted in a random attachment of tag moieties over the surface of the antibody. Modification with up to 6 to 12 NHS ester containing molecules per antibody molecule typically results in no decrease in antigen binding activity. Even higher ratios of NHS ester to antibody are possible with only slight loss of activity.

Protocol

1. Purified human IgG (purchased from Sigma-Aldrich) was diluted to 2 mg/ml in 1×PBS (0.1 M sodium phosphate, 0.15 M NaCl, pH 7.2).

2. NHS ester containing tag moieties was dissolved in DMF (dimethylformamide) to a final concentration between 10 to 20 nmols/□l DMF.

3. 500 μL of diluted human IgG (6.5 nmol) was mixed with either 1, 5, 25, or 50 μl of tag moiety (14, 68, 340, and 680 nmols respectively).

4. The solution was allowed to react for 2 hours on ice in the dark.

5. The tag moiety-conjugated antibody was purified by dialysis against 0.1×PBS (10 mM sodium phosphate, 15 mM NaCl, pH 7.2) for 20 hours at 4° C.

Sandwich Immunoassays for Cytokines

A sandwich-type immunoassay was carried out. The assay allows for the qualification and quantification of known cytokine antigens. In this assay, a matched pair of antibodies forms a sandwich around a cytokine antigen bringing the two antibodies in close proximity. One of these antibodies is conjugated with a tag moiety to yield a tagged probe. The tagged probes have a singlet oxygen labile linkage, which allows the release of the tag reporter after reaction with singlet oxygen. The second antibody is conjugated to a sensitizer dye that produces singlet oxygen when irradiated at 680 nm. Due to the relatively short half-life of the singlet oxygen, only when the two antibodies form a sandwich does the singlet oxygen cleave the cleavable linkage of the tagged probe.

Protocol for a Sandwich Immunoassay for Cytokines

1. 10 μl of assay buffer (0.1×PBS, 40 mg/ml BSA) is mixed with 1 μl (100 nM) of biotin-labeled anti-human IL-4 monoclonal antibody (purchased from Pierce, catalogue number M-450-B) and 1 μl of cytokine IL-4 (Pierce, catalogue number R-IL-4-5) ranging in concentration from 0 to 500 nM.

2. The reaction was allowed to proceed for 30 minutes at room temperature.

3. 5 μl of 100 μg/ml streptavidin-labeled sensitizer beads were added and the mixture was incubated for 15 minutes at room temperature in the dark.

4. To remove non-specific interactions of the tagged probes with streptavidin, 2 μl of 5 μM biotin-DNP was added and incubated for 10 minutes at room temperature in the dark. 1 μl of 400 nM anti-human IL-4 polyclonal antibody conjugated to an amino-dextran tag moiety was added and incubated for 30 minutes at room temperature in the dark.

5. The above procedure was repeated for various cytokines and various tag moieties as follows: IL-6 was studied using tag moiety Pro 10, IFNγ was studied using tag moiety Pro 8, TNFα was studied using tag moiety Pro 7, IL-10 was studied using tag moiety Pro 4, IL-8 was studied using tag moiety Pro 2. A multiplexed assay for six cytokines (IL-4, IL-6, IL-8, IL-10, TNFα, and IFNγ) was conducted.

6. The reaction mixture was then irradiated for 30 s using a 150 watt lamp source with a optical filter of 680 DF+20 nm. The released tags are separated using HPLC. Briefly, the sample is loaded through a Pierce guard column onto a C₁₈ column (particle size 3 μm, pore size 10 nm) in buffer A (0.1 M triethylammonium acetate, pH 7.0, 1% acetonitrile). Tag reporters are eluted by a linear gradient of acetonitrile, up to a concentration of 50% acetonitrile in buffer A.

A second buffer system useful for separating tag reporters on a C₁₈ column contains an ion pairing reagent, tetrabutylammonium hydrogen sulphate. The starting buffer is 50 mM potassium phosphate, pH 5.9, 2 mM tetrabutylammonium hydrogen sulphate, which is mixed with 50 mM potassium phosphate, pH 5.9, 2 mM tetrabutylammonium hydrogen sulphate, 60% acetonitrile to obtain a gradient of increasing concentration of acetonitrile.

Tag reporters eluted from the C₁₈ column are detected using a fluorescence detector.

All journal article, reference and patent citations provided above, in parentheses or otherwise, whether previously stated or not, are incorporated herein by reference in their entirety.

Although the invention has been described with reference to the disclosed embodiments, those skilled in the art will readily appreciate that the specific experiments detailed are only illustrative of the invention. It should be understood that various modifications can be made without departing from that spirit of the invention.

What is claimed is:

1. A method for detecting a target nucleic acid sequence, comprising:
(a) contacting one or more target nucleic acid sequences with a set of tagged probes under conditions sufficient for hybridization of a target nucleic acid sequence with a tagged probe, said tagged probes comprising a mobility modifier attached to a nucleic acid target binding moiety by a bond that is cleavable by a nuclease, said nucleic acid target binding moiety containing at least one bond resistant to said nuclease;
(b) treating the tagged probe hybridized to the target nucleic acid with a nuclease under conditions sufficient for cleavage of the nuclease-cleavable bond to release a tag reporter;
(c) separating said tag reporter using a chromatographic method, and (d) detecting a tag reporter corresponding to a known target sequence.

2. The method of claim 1, wherein said chromatographic method selected from the group consisting of reversed-phase chromatography, size exclusion chromatography, affinity chromatography and ion exchange chromatography.

3. The method of claim 1, wherein said one or more target nucleic acid sequences further comprise a plurality of different target nucleic acid sequences.

4. The method of claim 1, wherein said tagged probes further comprise a capture ligand having specific binding activity for a capture agent.

5. The method of claim 4, wherein said nucleic acid target binding moiety of the tagged probes further comprises a capture ligand having specific binding activity for a capture agent.

6. The method of claim 4, further comprising binding the set of tagged probes with a capture agent.

7. The method of claim 5, further comprising the step of separating one or more cleaved tagged probes from un-cleaved or partially cleaved tagged probes.

8. The method of claim 4, wherein said capture ligand further comprises biotin or an antigen.

9. The method of claim 4, wherein said capture agent is selected from the group consisting of avidin, streptavidin, an antibody, a receptor, or a functional fragment thereof, having specific binding activity to the capture ligand.

10. The method of claim 1, wherein said mobility modifier is linked to the nucleic acid target binding moiety by a phosphodiester bond.

11. The method of claim 1, wherein said nuclease-resistant bond is selected from the group consisting of thiophosphate, phosphinate, phosphoramidate, amide, and boronate bonds.

12. The method of claim 1, wherein said nuclease is an exonuclease.

13. The method of claim 1, wherein said nucleic acid target binding moiety further comprises a nucleic acid sequence that can specifically hybridize to a single nucleotide polymorphism in a nucleic acid target sequence.

14. A method for detecting a target analyte, comprising:
(a) contacting one or more target analytes with a set of tagged probes attached to a cleavage-inducing moiety under conditions sufficient for binding of a target analyte with a tagged probe, said tagged probes comprising a mobility modifier attached to a target binding moiety by a cleavable linkage, said cleavable linkage being susceptible to cleavage when said cleavage-inducing moiety is activated by visible light;
(b) separating tagged probes bound to a target binding moiety from unbound tagged probes;
(c) activating said cleavage-inducing moiety with visible light to release a tag reporter;
(d) separating said tag reporter using a chromatographic method, and (e) detecting a tag reporter corresponding to target analyte.

15. The method of claim 14, wherein said chromatographic method is selected from the group consisting of reversed-phase chromatography, size exclusion chromatography, affinity chromatography and ion exchange chromatography.

16. The method of claim 14, wherein said one or more target analytes further comprise a plurality of different target analytes.

17. The method of claim 14, wherein said one or more target analytes are selected from the group consisting of polypeptides, proteins, peptides, polysaccharides, nucleic acids, sugars, lipids, and small molecules.

18. The method of claim 14, wherein said cleavage-inducing moiety further comprises a sensitizer

19. The method of claim 14, wherein said cleavage-inducing moiety is a sensitizer capable of generating singlet oxygen.

20. The method of claim 18, wherein said cleavage-inducing moiety is a sensitizer selected from the group consisting of benzophenone, 9-thioxanthone, eosin, 9,10,-dibromoanthraene, methylene blue, metallo-porphyrins, chloroperoxidase and myeloperoxidase.

21. The method of claim 14, wherein said cleavage-inducing moiety further comprises two or more cleavage-inducing moieties.

22. The method of claim 14, wherein said cleavable linkage is susceptible to cleavage by singlet oxygen.

23. The method of claim 14, wherein said tagged probes further comprise a capture ligand having specific binding activity for a capture agent.

24. The method of claim 23, wherein said target binding moiety of the tagged probes further comprises a capture ligand having specific binding activity for a capture agent.

25. The method of claim 23, further comprising binding the set of tagged probes with a capture agent.

26. The method of claim 24, further comprising the step of separating one or more cleaved tagged probes from un-cleaved or partially cleaved tagged probes.

27. The method of claim 23, wherein said capture ligand further comprises biotin or an antigen.

28. The method of claim 23, wherein said capture agent is selected from the group consisting of avidin, streptavidin, an antibody, a receptor, or a functional fragment thereof, having specific binding affinity to the capture ligand.

29. A method for detecting a target analyte, comprising:
(a) contacting one or more target analytes with a set of first and second binding reagents under conditions sufficient for binding of a target analyte with said first and second binding reagents, each of said first binding reagents comprising a cleavage-inducing moiety and a target binding moiety, each of said second binding reagents comprising a tagged probe having a mobility modifier attached to a target binding moiety by a cleavable linkage, said cleavable linkage being susceptible to cleavage when in proximity to an activated cleavage-inducing moiety;
(b) activating said cleavage-inducing moiety to release a tag reporter;
(c) separating said tag reporter using a chromatographic method, and
(d) detecting a tag reporter corresponding to a known target analyte.

30. The method of claim 29, wherein said chromatographic property is detected using a chromatographic method selected from the group consisting of reversed-phase chromatography, size exclusion chromatography and ion exchange chromatography.

31. The method of claim 29, wherein said one or more target analytes further comprise a plurality of different target analytes.

32. The method of claim 29, wherein said one or more target analytes further comprise a binding partner of a specific binding pair.

33. The method of claim 29, wherein said one or more target analytes are selected from the group consisting of polypeptides, proteins, peptides, polysaccharides, nucleic acids, sugars, lipids, and small molecules.

34. The method of claim 29, wherein said first or second binding reagent further comprises a binding partner of a specific binding pair.

35. The method of claim 29, wherein said target binding moiety is selected from the group consisting of ligand, antiligand, receptor, antibody, biotin, avidin, strepavidin, protein A and polynucleotide.

36. The method of claim 29, wherein said cleavage-inducing moiety further comprises a photosensitizer or a chemi-activated sensitizer.

37. The method of claim 29, wherein said cleavage-inducing moiety is a sensitizer capable of generating singlet oxygen.

38. The method of claim 36, wherein said cleavage-inducing moiety is a sensitizer selected from the group consisting of benzophenone, 9-thioxanthone, eosin, 9,10,-dibromoanthraene, methylene blue, metallo-porphyrins, chloroperoxidase and myeloperoxidase.

39. The method of claim 29, wherein said cleavage-inducing moiety further comprises two or more cleavage-inducing moieties.

40. The method of claim 29, wherein said cleavable linkage is susceptible to cleavage by singlet oxygen.

41. The method of claim 29, wherein said tagged probes further comprise a capture ligand having specific binding activity for a capture agent.

42. The method of claim 41, wherein said target binding moiety of the tagged probes further comprises a capture ligand having specific binding activity for a capture agent.

43. The method of claim 41, further comprising binding the set of tagged probes with a capture agent.

44. The method of claim 42, further comprising the step of separating one or more cleaved tagged probes from un-cleaved or partially cleaved tagged probes.

45. The method of claim 41, wherein said capture ligand further comprises biotin or an antigen.

46. The method of claim 41, wherein said capture agent is selected from the group consisting of avidin, streptavidin, an antibody, a receptor, or a functional fragment thereof, having specific binding affinity to the capture ligand.

47. A method for identifying a binding partner of a specific binding pair, comprising:
(a) incorporating a cleavage-inducing moiety into a first binding partner of a specific binding pair;
(b) contacting said first binding partner having an incorporated cleavage-inducing moiety with a set of second binding partners under conditions sufficient for binding, each of said second binding partners comprising a tagged probe having a mobility modifier attached to a target binding moiety by a cleavable linkage, said cleavable linkage being susceptible to cleavage when in proximity to an activated cleavage-inducing moiety;
(c) activating said cleavage-inducing moiety to release a tag reporter;
(d) separating said tag reporter using a chromatographic method, and
(e) detecting a tag reporter corresponding to a known second binding partner of a specific binding pair.

48. The method of claim 47, wherein said chromatographic method is selected from the group consisting of reversed-phase chromatography, size exclusion chromatography, affinity chromatography and ion exchange chromatography.

49. The method of claim 47, wherein said first binding partner further comprises a plurality of different first binding partners.

50. The method of claim 47, wherein said first binding partner further comprises a ligand, antiligand, nucleic acid, or a functional fragment thereof.

51. The method of claim 47, wherein said first binding partner is selected from the group consisting of polypeptides, proteins, peptides, polysaccharides, nucleic acids, sugars, lipids, and small molecules.

52. The method of claim 47, wherein said second binding partner further comprises a ligand, antiligand, nucleic acid, or a functional fragment thereof.

53. The method of claim 47, wherein said target binding moiety further comprises said second binding partner.

54. The method of claim 47, wherein said target binding moiety further comprises a moiety that specifically binds to said second first binding partner.

55. The method of claim 54, wherein said target binding moiety is selected from the group consisting of ligand, antiligand, receptor, antibody, biotin, avidin, strepavidin, protein A and polynucleotide, or a functional fragment thereof.

56. The method of claim 47, wherein said cleavage-inducing moiety further comprises a photosensitizer or a chemi-activated sensitizer.

57. The method of claim 47, wherein said cleavage-inducing moiety is a sensitizer capable of generating singlet oxygen.

58. The method of claim 56, wherein said cleavage-inducing moiety is a sensitizer selected from the group consisting of benzophenone, 9-thioxanthone, eosin, 9,10,-dibromoanthraene, methylene blue, metallo-porphyrins, chloroperoxidase and myeloperoxidase.

59. The method of claim 47, wherein said cleavage-inducing moiety further comprises two or more cleavage-inducing moieties.

60. The method of claim 47, wherein said cleavable linkage is susceptible to cleavage by singlet oxygen.

61. The method of claim 47, wherein said tagged probes further comprise a capture ligand having specific binding activity for a capture agent.

62. The method of claim 61, wherein said target binding moiety of the tagged probes further comprises a capture ligand having specific binding activity for a capture agent.

63. The method of claim 61, further comprising binding the set of tagged probes with a capture agent.

64. The method of claim 62, further comprising the step of separating one or more cleaved tagged probes from un-cleaved or partially cleaved tagged probes.

65. The method of claim 61, wherein said capture ligand further comprises biotin or an antigen.

66. The method of claim 61, wherein said capture agent is selected from the group consisting of avidin, streptavidin, an antibody, a receptor, or a functional fragment thereof, having specific binding affinity to the capture ligand.
